(19) United States
(12) Patent Application Publication
(10) Pub. No.: US 2013/0142734 A1
(43) Pub. Date: Jun. 6, 2013

(54) TARGETED DELIVERY SYSTEMS FOR DIAGNOSTIC APPLICATIONS

(71) Applicant: Ayelet David, Omer (IL)

(72) Inventor: Ayelet David, Omer (IL)

(21) Appl. No.: 13/521,345

(22) PCT Filed: Jan. 11, 2011
(86) PCT No.: PCT/IL11/00029

§ 371 Date: Dec. 18, 2012

Related U.S. Application Data
(60) Provisional application No. 61/294,186, filed on Jan. 12, 2010.

(51) Int. Cl. A61K 49/00

(52) U.S. Cl. CPC A61K 49/0054
USPC 424/9.6; 525/54.1; 525/54.2

(57) ABSTRACT

Targeting of imaging probes specifically to diseased tissues such as cancer is attractive because it potentially allows the improvement of tumor detection. One of the problems associated with conventional, low molecular weight imaging probes is the limited tumor:background ratio. To circumvent this, imaging probes may be conjugated to polymeric carriers to target solid tumors by either passive accumulation of macromolecules into tumor tissues due to the “enhanced permeability and retention” effect (EPR effect) or active targeting through the incorporation of cell-specific recognition ligands that mediate binding to cancer-specific antigens. This invention describes an innovative targeting strategy for the selective delivery of diagnostic agents into solid tumors by means of polymer-NIR fluorochrome conjugates modified with targeting ligands that bind to antigens or receptors that are uniquely expressed or over-expressed on the target cells relative to normal tissues.

### FIELD OF THE INVENTION

This invention describes a targeting strategy for the selective delivery of diagnostic agents to cells by means of polymer-chromophore conjugates modified to include targeting ligand which enhances the specificity and/or sensitivity of the diagnostic agent.

### BACKGROUND OF THE INVENTION

Optically based biomedical imaging techniques have advanced over the past decade due to factors including developments in laser technology, sophisticated reconstruction algorithms and imaging software originally developed for non-optical, tomographic imaging modes such as CT and MRI. Visible wavelengths are used for optical imaging of surface structures by means of endoscopy and microscopy.

Near infrared wavelengths (approx. 700-1000 nm) have been used in optical imaging of internal tissues, because near infrared radiation exhibits tissue penetration of up to 6-8 centimeters. See, e.g., Wyatt, 1997, “Cerebral oxygenation and haemodynamics in the fetus and newborn infant,” Phil. Trans. R. Soc. London B 352:701-706; Tromberg et al., 1997, “Non-invasive measurements of breast tissue optical properties using frequency-domain photo migration,” Phil. Trans. R. Soc. London B 352:661-667.

Advantages of near infrared imaging over other currently used clinical imaging techniques include the following: potential for simultaneous use of multiple, distinguishable probes (important in molecular imaging); high temporal resolution (important in functional imaging); high spatial resolution (important in in vivo microscopy); and safety (no ionizing radiation).

In near infrared fluorescence imaging, filtered light or a laser with a defined bandwidth is used as a source of excitation light. The excitation light travels through body tissues. When it encounters a near infrared fluorescent molecule (“contrast agent”), the excitation light is absorbed. The fluorescent molecule then emits light (fluorescence) spectrally distinguishable (slightly longer wavelength) from the excitation light. Despite good penetration of biological tissues by near infrared light, conventional near infrared fluorescence probes are subject to many of the same limitations encountered with other contrast agents, including low target/background ratios.

There remains a need for effective targeting of cancerous cells and tissue and thereby an effective cancer diagnostic and others.

### SUMMARY OF THE INVENTION

In one embodiment this invention provides a polymer characterized by the structure of formula 1:

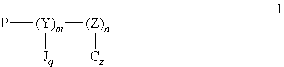

wherein
m, n, q and z indicate percentages of the respective monomer composition of the polymer, wherein m is between about 0.05%-50%, n is between 0.5 to 50%; and q and z are between about 0.5%-50%
C is a near infrared dye selected from the group consisting of Cy5, Cy5.5 Indocyanine green (ICG), IR783 and analogs thereof, covalently linked to the polymeric backbone.
J is a short peptide, antibody fragment, monosaccharide or oligosaccharide targeting moiety;
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm comprises a protease-cleavable linker, a pH-sensitive linker or an esterase-cleavable linker; and
P is a polymeric group comprising underivatized or derivatized monomers of N-(2-hydroxypropyl)methacrylamide (HPMA), N-methylacrylamide, N,N-dialkylacrylamides, acrylic acid, methacrylic acid, polyamino acids, polysaccharides, polymers containing poiyethyleneoxide sequences and polyvinyl pyrrolidone-maleic anhydride polymers, polylactic-co-glycolic acid, dendrimers, polysaccharides, peptides, proteins, polymer-peptide conjugates or polymer-protein conjugates.

In one embodiment, this invention provides a polymer represented by the structure of formula III:

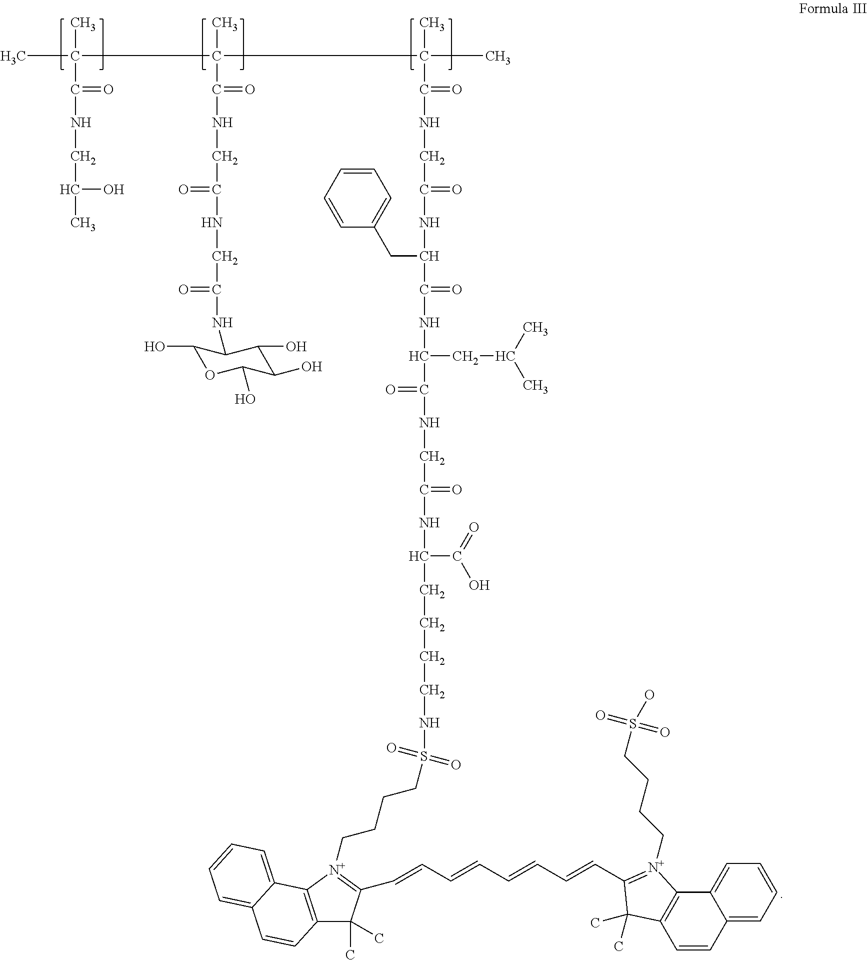

In one embodiment, this invention provides a polymer represented by the structure of formula IV:

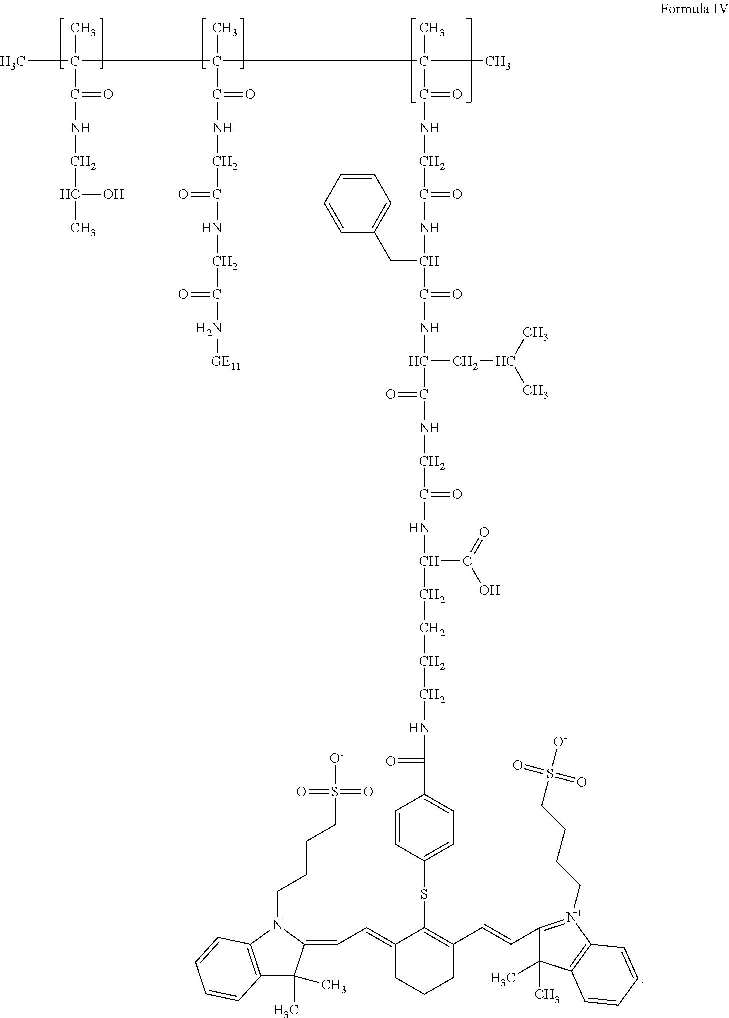

In some embodiments, the invention provides a pharmaceutical composition comprising a polymer of this invention.

In some embodiments, the invention provides a method of imaging an inflammatory condition in a subject, said method comprising administering a polymer of this invention to said subject.

In some embodiments, the invention provides a method of imaging a disease associated with neovascularization in a subject, said method comprising administering a polymer of this invention to said subject.

In some embodiments, the invention provides a method of imaging a cancer or cancerous tissue in a subject, said method comprising the step of contacting said cancer or cancerous tissue with a polymer of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the emission spectrum for NIR Dyes (ICG, IR-783, and 783-S-Ph-COOH) following excitation at 650 nm (A) and 690 nm (B).

FIG. 2A depicts the fluorescence intensity of the NIR dyes at various concentrations, and absorption spectrum of the NIR dyes is shown in FIG. 2B. FIG. 2C depicts the effect of IR-783-S-Ph-COOH loading on the quenching efficiency of P-GGFLGK-IR783/

FIG. 3 depicts the effect of NIR783 loading on p-HPMA-NIR783 Quenching Efficiency.

FIG. 4A depicts fluorescence intensity following p-HPMA-GFLGK-IR-783 in vitro degradation by Cathepsin B. FIG. 4B depicts the optical activation of different IR783 labeled copolymer by CB enzyme.

FIG. 5 depicts peptide characterization using HPLC and MALDI-TOF/

FIG. 6 depicts whole body image of orthotopically implanted tumors in mice 4 h post injection of 2 mg of P-(GGFLGK-IR783)7.5% copolymer and ex vivo imaging of major organs at this time point.

FIG. 7 depicts whole body image of orthotopically implanted tumors in mouse 4, 24 and 48 h post injection of 2 mg P-(GGFLGK-IR783)2.5% copolymer and ex vivo imaging of major organs 48 h after injection.

FIG. 8A depicts whole body image rectally implanted tumors in mouse 4 and 24 h post injection of 0.2 mg P-(GGFLGK-IR783)2.5% copolymer and ex vivo imaging of major organs 24 h after injection. FIG. 8B depicts whole body image rectally implanted tumors in mouse 4 and 24 h post injection of 0.2 mg P-(GGFLGK-IR783)7.5% copolymer and ex vivo imaging of major organs 48 h post injection.

FIG. 9A depicts whole body image of HT29 rectally implanted tumors in mouse 4, 24 and 48 h post injection of 1 mg P-(GGFLGK-IR783)7.5% copolymer. FIG. 9B depicts ex vivo imaging of major organs 48 h after injection of 1 mg P-(GGFLGK-IR783)7.5% copolymer.

FIG. 10A depicts whole body image of HT29 rectally implanted tumors in mouse 4, 24 and 48 h post injection of 1 mg P-(GGFLGK-IR783)7.5% copolymer. FIG. 10B depicts the average fluorescence efficiency in excised organs 48 h post injection of 1 mg P-(GGFLGK-IR783)7.5% copolymer.

FIG. 11 depicts whole body image of rectally implanted tumors mouse 4, 24 and 48 h post injection of 1 mg P-GE11-(GGFLGK-IR783) copolymer and ex vivo imaging of major organs 48 h after injection.

FIG. 12 depicts whole body image of rectally implanted tumors mouse 4, 24 and 48 h post injection of 0.2 mg P-(GGFLGK (SEQ ID NO: 11)-IR783)7.5% copolymer and ex vivo imaging of major organs 48 h after injection.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention provides, inter alia, for the specific targeting of imaging agents.

In one embodiment this invention provides a polymer characterized by the structure of formula 1:

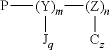

wherein
m, n, q and z indicate percentages of the respective monomer composition of the polymer, wherein m is between about 0.05%-50%, n is between 0.5 to 50%; and q and z are between about 0.5%-50%
C is an a near infrared dye selected from the group consisting of Cy5, Cy5.5 Indocyanine green (ICG), IR783 and analogs thereof, covalently linked to the polymeric backbone
J is a short peptide, monosaccharide or oligosaccharide targeting moiety;
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm is a protease-cleavable linker, a pH-dependent cleavable linker or an esterase-cleavable linker; and
P is a polymeric group comprising underivatized or derivatized monomers of N-(2-hydroxypropyl)methacrylamide (HPMA), N-methylacrylamide, N,N-dialkylacryl amides, acrylic acid, methacrylic acid, polyamino acids, polysaccharides, polymers containing polyethyleneoxide sequences and polyvinyl pyrrolidone-maleic anhydride polymers, polylactic-co-glycolic acid, dendrimers, polysaccharides, peptides, proteins, polymer-peptide conjugates or polymer-protein conjugates.

In one embodiment the invention provides a polymer of formula 1 wherein the molecular weight of the polymer ranges between 100 Da and 1000 kDa. In one embodiment the molecular weight of the polymer is less than 60 kDa. In one embodiment, the molecular weight of the polymer ranges between 15-60 kDa. It will be appreciated by the skilled artisan that molecular weight may vary as a function of the particular monomers chosen, and that such variations are to be considered as part of this invention.

In one embodiment the composition comprising polymer of formula 1 is about 80 molar % of Y and Z and about 20 molar % of J and C.

In another embodiment Y is characterized by the structure of formulae IIa, or IIb or IIc as follows:

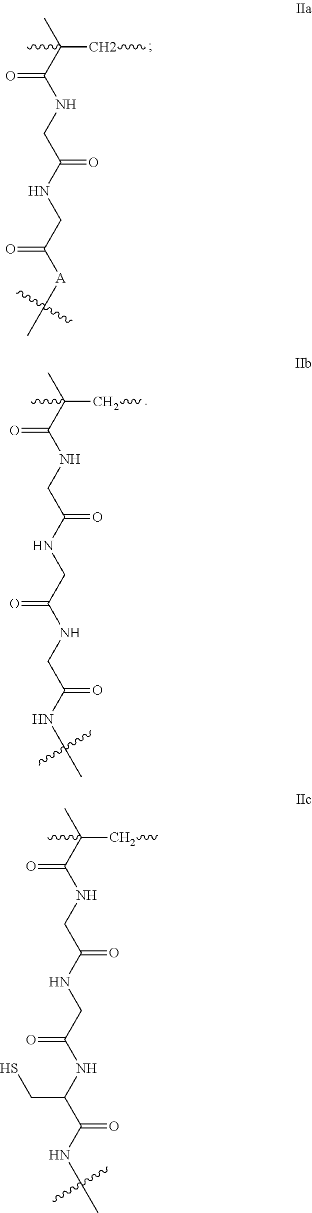

In some embodiments, according to this aspect, A is an amine or alcohol.

In one embodiment the polymer is represented by the structure of formula III:

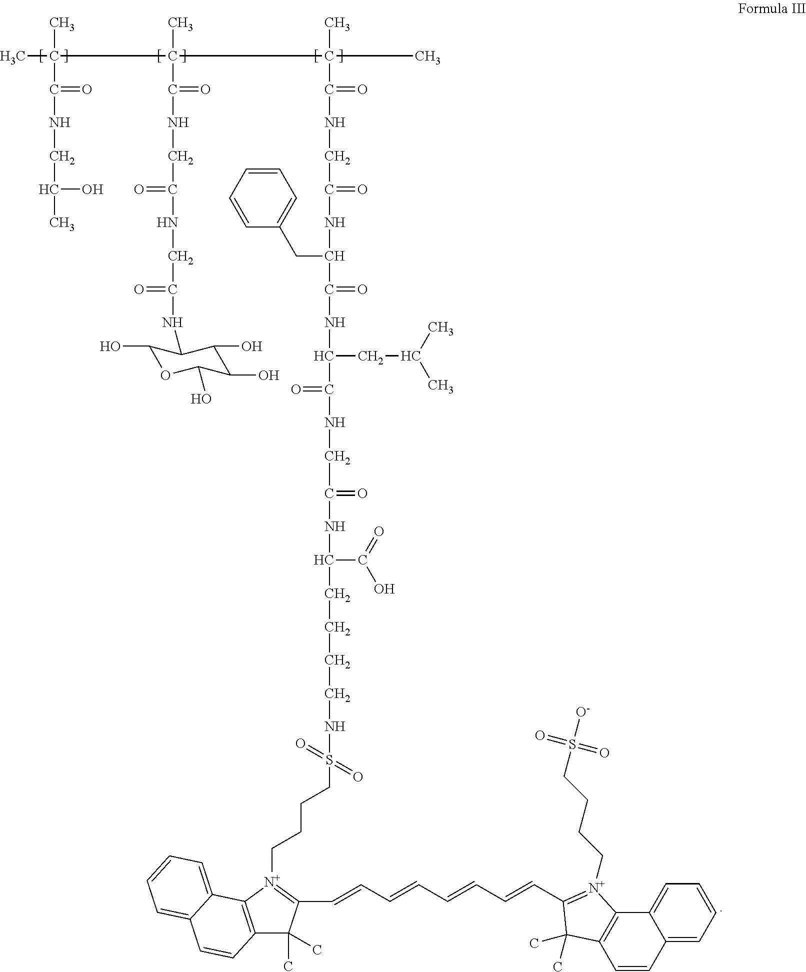 [SMILES: CC(C)CC(NC(=O)C(Cc1ccccc1)NC(=O)CNC(=O)C(C)(C)C(C)(C(=O)NCC(=O)NCC(=O)NC1C(O)OC(O)C(O)C1O)C(C)(C)C(=O)NCC(C)O)C(=O)NCC(=O)NC(CCCCNS(=O)(=O)CCCC[N+]1=C(/C=C/C=C/C=C/C=C/C2=[N+](CCCCS(=O)(=O)[O-])c3c(ccc4ccccc34)C2(C)C)C(C)(C)c2ccc3ccccc3c21)C(=O)O]

In some embodiments, the polymer is represented by the structure of formula IV:

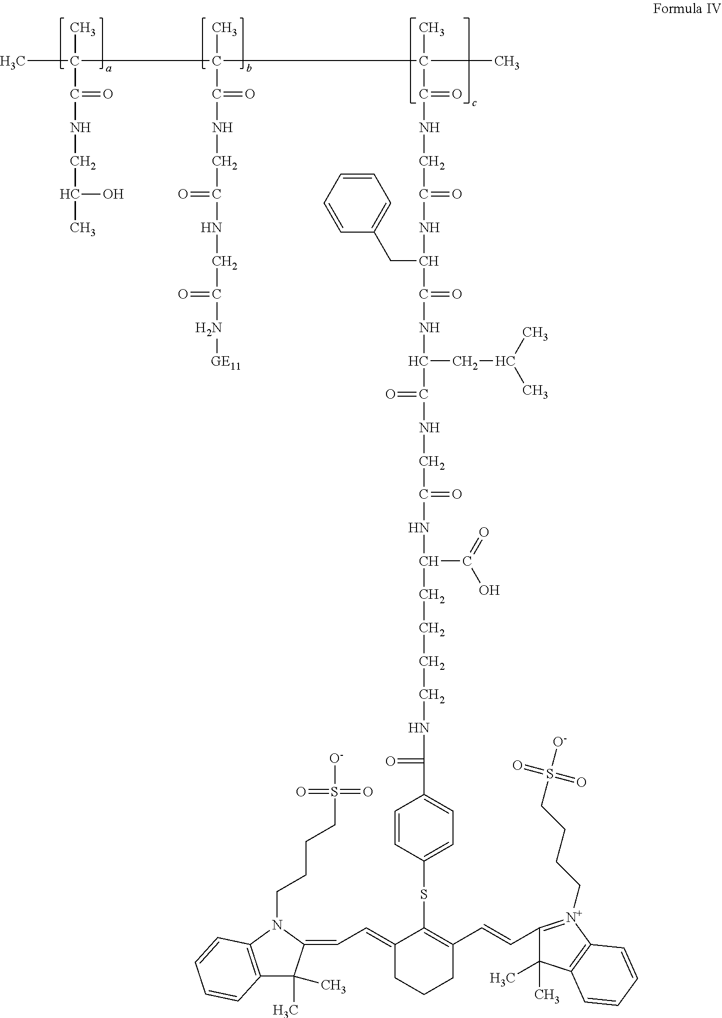

In some embodiments, Z is a protease cleavable linker, which is cleavable by a lysosomal thiol-dependent protease or in some embodiments the protease cleavable linker is a tetra-peptide degradable spacer. In some embodiments, the linker comprises the sequence GFLG (SEQ ID NO: 1); GGGGFG (SEQ ID NO: 2); GGGFLG (SEQ ID NO: 3); GGEE (SEQ ID NO: 4); GGGLFG (SEQ ID NO: 5) or GGKK (SEQ ID NO: 6).

In some embodiments, Z is a pH-sensitive cleavable linker, which in some embodiments comprises a cis aconityl, acetal or hydrazone moiety which undergoes pH-dependent hydrolysis following internailization within an acidic intracellular compartment.

In some embodiments, the invention contemplates use of a non-cleavable linker for Z.

In some embodiments, J is a short peptide or monosaccharide or oligosaccharide carbohydrate targeting moiety. In some embodiments, the carbohydrate targeting moiety is a monosaccharide, an oligosaccharide or a derivative thereof.

In some embodiments, the term “short peptide” refers to peptides of 3-15 amino acids in length.

In one embodiment, J is a peptide having the sequence YHWYGYTPQNVI (SEQ ID NO: 7) or ANTPCGPYTHDCPVKR (SEQ ID NO: 8).

In some embodiments, the peptide targeting moiety is a monoclonal antibody or a fragment thereof, which binds to a specific cell surface marker and in some embodiments, the cell surface marker is a cancer marker.

In some embodiments, the targeting ligand increases selectivity/specificity of the agent for the selected cells, thereby enhancing the sensitivity of the diagnostic.

Targeting of imaging probes specifically to diseased tissues is associated with a limited tumor:background ratio. In one embodiment of this invention, the conjugation of imaging probes to polymeric carriers to target solid tumors is improved over traditional methods, which do not employ such targeting ligands and instead rely upon passive accumulation of macromolecules into tumor tissues due to the “enhanced permeability and retention” effect (EPR effect). In one embodiment, this invention provides an innovative targeting strategy for the selective delivery of diagnostic agents into solid tumors by means of polymer-NIR fluorochrome conjugates modified with targeting ligands that bind to antigens or receptors that are uniquely expressed or over-expressed on the target cells relative to normal tissues.

In some embodiments, the targeting moiety will be a lectin or galectin. In some embodiments, the lectin is an endogenous lectin. Endogenous (also called animal) lectins are a class of glycoproteins that have specific and non-covalent binding sites for defined carbohydrates. The expression of endogenous lectins on cancer cells depends upon the cell type, cell differentiation state, cell metastatic potential, cell oncogene expression and cell anatomical growth site and endogenous surface lectins of malignant cells participate in the process of tumor cell growth regulation and in their metastatic spread. The invention therefore contemplates incorporation of an endogenous lectin, or fragment thereof, as a targeting moiety. Such endogenous lectins may include, but are not limited to the asialoglycoprotein receptor (ASGP-R), galectins (galectin 1, galectin 3), selectins (E-selectin, P-selectin), mannose receptors (ManR, mannose-binding protein (MBP)) and hyaluronic acid receptors (CD44, receptor for hyaluronan-mediated motility (RHAMM)).

In some embodiments, galectins, also referred to as S-type (sulfhydryl-dependent) galactoside-binding lectins, are contemplated according to this aspect. In some embodiments, melanomas, astrocytomas, and bladder and ovarian tumors overexpress various galectins, and heightened galectin expression (especially galectin-1, and galectin-3) usually correlates with clinical aggressiveness of the tumor and the progression to a metastatic phenotype, supporting their incorporation as a targeting moiety within the claimed polymers of this invention.

In some embodiments, the targeting moiety is a ligand for the epidermal growth factor receptor (EGFR). According to this aspect, and in one embodiment, such targeting moiety may include a peptide having a sequence YHWYGYTPQNVI (SEQ ID NO: 9) designated as GE11, which specifically binds to EGFR.

In some embodiments, specific use of an agent, which undergoes quenching, when the agent is not in the desired cellular compartment, allows for enhanced assay sensitivity, as well, as will be appreciated by the skilled artisan.

In some embodiments, according to this aspect, m, n, q and z indicate percentages of the respective monomer composition of the polymer, wherein m is between about 0.05%-50%, n is between 0.5 to 50%; and q and z are between about 0.5%-50%.

In some embodiments, according to this aspect, the imaging agent is indocyanine green (ICG), or 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide (IR783).

In some embodiments, with reference to the polymers of this invention, the term “alkane” refers, for example, to branched and unbranched molecules having the general formula CₙH₂ₙ₊₂, wherein n is, for example, a number from 1 to about 100 or more, such as methane, ethane, n-propane, isopropane, n-butane, isobutane, tert-butane, octane, decane, tetradecane, hexadecane, eicosane, tetracosane, and the like. Alkanes may be substituted by replacing hydrogen atoms with one or more functional groups. The term “aliphatic” refers, for example, to straight-chain molecules, and may be used to describe acyclic, unbranched alkanes. The term “long-chain” refers, for example, to hydrocarbon chains in which n is a number of from about 8 to about 60, such as from about 20 to about 45 or from about 30 to about 40. The term “short-chain” refers, for example, to hydrocarbon chains in which n is an integer of from about 1 to about 7, such as from about 2 to about 5 or from about 3 to about 4.

In some embodiments, with reference to the polymers of this invention, the term “alkene” refers to any open chain hydrocarbon having carbon to carbon double bonds, wherein each of the carbons containing at least one of the double bonds is joined to either hydrogen or another carbon. Alkenes include compounds having more than one double bond.

In one embodiment, with reference to the polymers of this invention, the alkanes or alkenes may be “substituted”, which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amine, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

In one embodiment, the term “amine” refers to any amine, including primary, secondary, tertiary, quaternary, or a combination thereof, as applicable herein.

In one embodiment, the term “protein” refers to large organic compounds made of amino acids arranged in a linear chain and joined together by peptide bonds between the carboxyl and amino groups of adjacent amino acid residues. In one embodiment the protein is made up of peptide segments. In one embodiment “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), *-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

In one embodiment, the term “amino acid” or “amino acids” is understood to include the naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

Peptides or proteins of this invention may be prepared by various techniques known in the art, including phage display libraries [Hoogenboom and Winter, J. Mol. Biol. 227:381 (1991); Marks et al., J. Mol. Biol. 222:581 (1991)].

In one embodiment the term “sugar” refers to a class of carbohydrate molecules including sucrose, lactose, and fructose. In one embodiment the term “sugar” represents a saccharide. In one embodiment the term “saccharide” is synonym with the term sugar. In one embodiment saccharide refers to a monosaccharide, disaccharide, oligosaccharide or polysaccharide. In one embodiment the monosaccharide has the molecular formula (CH₂O)n. In one preferred embodiment the monosaccharide is a molecule having the molecular formula C₆H₁₂O₆. In one embodiment monosaccharides comprise glucose (dextrose), fructose, galactose, xylose and ribose. In some embodiments, disaccharides comprise sucrose (common sugar) and polysaccharides (such as cellulose and starch).

In one embodiment, the sugar is a sugar derivative. The term sugar derivative refers to any compound being derived from a sugar. In the present context sugar means any carbohydrate, including monosaccharides, disaccharides, trisaccharides, oligosaccharides, and polysaccharides, whether being a five-membered ring (pentose) or a six-membered ring (hexose) or combinations thereof, or whether being a D-form or an L-form, as well as substances derived from monosaccharides by reduction of the carbonyl group (alditols), by oxidation of terminal groups to carboxylic acids, or by replacement of hydroxy groups by another group. It also includes derivatives of these compounds. Examples of derivatives of the sugars are uronic acids, aldoses, in which the first CH₂OH-group has been exchanged with a carboxy group; aldaric acids, aldonic acids, in which the first CH₂OH-group has been exchanged with a carboxy group; deoxy sugars, monosaccharides, in which a hydroxyl group has been exchanged with a hydrogen; amino sugars, monosaccharides, in which a hydroxyl group has been exchanged with an amino group.

In one embodiment R₁, R₂, R₃, R₄, R₁′, R₂′, R₃′ and R₄ comprise a synthetic polymer. the term “synthetic polymer” refers to resins and polymers including polymethylmethacrylate (PMMA), acrylics, acrylates, polyethylene, polyethylene terepthalate, polycarbonate, polystyrene and other styrene polymers, polypropylene, polytetrafluoroethylene. In one embodiment, the polymers of this invention are polymers. In another embodiment, the polymers of this invention are homo- or, in another embodiment heteropolymers. In another embodiment, the polymers of this invention are synthetic, or, in another embodiment, the polymers are natural polymers. In another embodiment, the polymers of this invention are free radical polymers, or, in another embodiment, graft polymers. In one embodiment, the polymers may comprise proteins, peptides or nucleic acids.

In one embodiment, this invention provides a polymer of formula I, III, IV, V, VI and/or an analog, derivative, isomer, metabolite, pharmaceutically acceptable salt, pharmaceutical product, hydrate, N-oxide, prodrug, polymorph, impurity or crystal or combinations thereof.

In one embodiment, this invention provides an analog of the polymer. In another embodiment, this invention provides a derivative of the polymer. In another embodiment, this invention provides an isomer of the polymer. In another embodiment, this invention provides a metabolite of the polymer. In another embodiment, this invention provides a pharmaceutically acceptable salt of the polymer. In another embodiment, this invention provides a pharmaceutical product of the polymer. In another embodiment, this invention provides a hydrate of the polymer. In another embodiment, this invention provides an N-oxide of the polymer. In another embodiment, this invention provides a prodrug of the polymer.

In another embodiment, this invention provides a composition comprising a polymer, as described herein, or, in another embodiment, a combination of an analog, derivative, isomer, metabolite, pharmaceutically acceptable salt, pharmaceutical product, hydrate, N-oxide, prodrug, polymorph, impurity or crystal of the polymers of the present invention.

In one embodiment, the term “isomer” includes, but is not limited to, optical isomers and analogs, structural isomers and analogs, conformational isomers and analogs, and the like.

In one embodiment, the term “isomer” is meant to encompass optical isomers of the polymer. It will be appreciated by those skilled in the art that the polymer of the present invention contain at least one chiral center. Accordingly, the polymer used in the methods of the present invention may exist in, and be isolated in, optically-active or racemic forms. Some compounds may also exhibit polymorphism. It is to be understood that the present invention encompasses any racemic, optically-active, polymorphic, or stereoisomeric form, or mixtures thereof, which form possesses properties useful in the treatment of androgen-related conditions described herein. In one embodiment, the polymer are the pure (R)-isomers. In another embodiment, the polymers are the pure (S)-isomers. In another embodiment, the polymers are a mixture of the (R) and the (S) isomers. In another embodiment, the polymers are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes “pharmaceutically acceptable salts” of the polymer of this invention, which may be produced, in one embodiment, using an amino-substituted polymer and an organic and inorganic acids, for example, citric acid and hydrochloric acid. Pharmaceutically acceptable salts can be prepared, from the phenolic compounds, in other embodiments, by treatment with inorganic bases, for example, sodium hydroxide. In another embodiment, esters of the phenolic compounds can be made with aliphatic and aromatic carboxylic acids, for example, acetic acid and benzoic acid esters. As used herein, “pharmaceutically acceptable salt” refers to, in one embodiment, those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M Berge, et al. describe pharmaceutically acceptable salts in detail in J. Pharmaceutical Sciences, 1977, 66: 1-1.9. The salts can be prepared in situ during the final isolation and purification of the compounds of the invention, or separately by reacting the free base function with a suitable organic acid. Representative acid addition salts include acetate, adipate, alginate, ascorbate, aspartate, benzene-sulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary as ammonium, and mine cations, including, but not limited to ammonium, tetramethyl ammonium, tetraethylammonium, methylamine, dimethylamine, trimethylamine, triethylamine, ethylamine, and the like.

The invention also includes N-oxides of the amino substituents of the polymer described herein.

This invention provides derivatives of the polymers. In one embodiment, “derivatives” includes but is not limited to ether derivatives, acid derivatives, amide derivatives, ester derivatives and the like. In another embodiment, this invention further includes hydrates of the polymers. In one embodiment, “hydrate” includes but is not limited to hemihydrate, monohydrate, dihydrate, trihydrate and the like.

This invention provides, in other embodiments, metabolites of the polymers. In one embodiment, “metabolite” means any substance produced from another substance by metabolism or a metabolic process.

This invention provides, in other embodiments, pharmaceutical products of the polymers of this invention. The term “pharmaceutical product” refers, in other embodiments, to a composition suitable for pharmaceutical use (pharmaceutical composition), for example, as described herein.

In some embodiments, the polymers of this invention comprise a ligand for a biological target, which in another embodiment, provides for directional specificity to cells or tissues. In one embodiment, the term “ligand for a biological target” refers to a molecule which enables the specific delivery of the polymer or composition of this invention to a particular site in vivo. In some embodiments, the phrase “targeting moiety” is synonymous therewith.

In one embodiment, the targeting agent specifically binds, or preferentially binds, only diseased cells, which in some embodiments, are vasculature-associated cells, for the effective and selective imaging of such cells.

In one embodiment, the polymeric group (P) comprises underivatized or derivatized monomers. In another embodiment, a derivatized monomer refers to a substituted monomer. In another embodiment, the monomer is substituted by an alkyl, halogen, cyano, nitro, amine, phosphonate or any combination thereof. In another embodiment, the monomer is substituted by another monomer forming a copolymer. In another embodiment, derivatized monomer refers to hydrolyzed, oxidized or reduced form of a monomer.

In one embodiment, with regard to P comprising derivatized monomers of N-(2-hydroxypropyl)methacrylamide (HPMA), N-methylacrylamide, N,N-dialkylacrylamides, acrylic acid, methacrylic acid, polyamino acids, polysaccharides, polymers containing polyethyleneoxide sequences and polyvinyl pyrrolidone-maleic anhydride polymers, polylactic-co-glycolic acid, dendrimers, saccharides, peptides, proteins, polymer-peptide conjugates and polymer-protein conjugates, it is to be understood that P may represent a copolymer of any combination of monomeric units as described in any repeating pattern, or any plausible or desired combination.

In one embodiment, the spacer is selected depending upon the properties desired. For example, the length of the spacer can be chosen to optimize the kinetics and specificity of ligand binding, including any conformational changes induced by binding of the ligand to a target receptor. The spacer, in some embodiments, should be long enough and flexible enough to allow the ligand moiety and the target cell receptor to freely interact. In some embodiments, if the spacer is too short or too stiff, there may be steric hindrance between the ligand moiety and the cell toxin.

In some embodiments, the spacer can be attached to the monomeric units comprising the polymer, using numerous protocols known in the art, such as those described in, for example, Pierce Chemicals “Solutions, Cross-linking of Proteins: Basic Concepts and Strategies,” Seminar #12, Rockford, Ill., and modifications of such methods may be readily achieved, as will be appreciated by the skilled artisan.

In some embodiments, several linkers may be included in order to take advantage of desired properties of each linker. Chemical linkers and peptide linkers may be inserted by covalently coupling the linker to the targeting agent (TA) and the imaging agent, for example. Heterobifunctional agents may be used to effect such covalent coupling. Peptide linkers may also be used. Flexible linkers and linkers that increase solubility of the polymers are contemplated for use, either alone or with other linkers are also contemplated herein.

In some embodiments, cleavable spacers are used. Heterobifunctional cleavable cross-linkers may comprise N-succinimidyl (4-iodoacetyl)-aminobenzoate; sulfosuccinimydil (4-iodoacetyl)-aminobenzoate; 4-succinimidyl-oxycarbonyl-a-(2-pyridyldithio)-toluene; sulfosuccinimidyl-6-[a-methyl-a-(pyridyldithiol)-toluamido]hexanoate; N-succinimidyl-3-(−2-pyridyldithio)-proprionate; succinimidyl 6[3(−(−2-pyridyldithio)-proprionamido]hexanoate; sulfosuccinimidyl 6[3(+2-pyridyldithio)-propionamido]hexanoate; 3-(2-pyridyldithio)-propionyl hydrazide, Ellman's reagent, dichlorotriazinic acid, S-(2-thiopyridyl)-L-cysteine. Further exemplary bifunctional spacers are disclosed in U.S. Pat. Nos. 5,349,066. 5,618,528, 4,569,789, 4,952,394, and 5,137,877.

The term linker and spacer may, in some embodiments, be considered to be synonymous.

Acid cleavable spacers, photocleavable and heat sensitive spacers may also be used, particularly where it may be necessary to cleave the targeted agent to permit it to be more readily accessible to reaction. Acid cleavable linkers/spacers include, but are not limited to, bismaleimideothoxy propane; and adipic acid dihydrazide linkers (see, e.g., Fattom et al. (1992) Infection &Immun. 60:584-589) and acid labile transferrin conjugates that contain a sufficient portion of transferrin to permit entry into the intracellular transferrin cycling pathway (see, e.g., Welhner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107, which linkers are herein incorporated by reference), thereby releasing the targeted agent upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable polymers, including hydroxypropylmethacrylamide polymer, glycine polymer, fluorescein polymer and methylrhodamine polymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

In some embodiments, such targeting polymers are characterized by of the polymers of this invention.

In one embodiment, the term “a tag” or “a labeling agent” refers to a molecule which renders readily detectable that which is contacted with a tag or a labeling agent. In one embodiment, the tag or the labeling agent is a marker polypeptide. In another embodiment, the labeling agent may be conjugated to another molecule which provides greater specificity for the target to be labeled. For example, and in one embodiment, the labeling agent is a fluorochrome conjugated to an antibody which specifically binds to a given target molecule, or in another embodiment, which specifically binds another antibody bound to a target molecule, such as will be readily appreciated by one skilled in the art.

In one embodiment imaging or detection is referred to as radiological. In one embodiment imaging or detection is done by means of an endoscope, for example, as descrbied in Gahlen et al. (1999) J. Photochem. Photobiol. B. 52:131-5; Major et al., 1997, Gynecol. Oncol. 66:122-132, and others.

In one embodiment imaging or detection is done by means of a catheter based device, including fiber optics devices, for example, as described in Tearney et al. 1997, Science 276: 2037-2039; Proc. Natl. Acad. Sci. USA 94:4256-4261.

In other embodiments, any appropriate imaging technology may be used, for example, phased array technology (Boas et al. 1994 Proc. Natl. Acad. Sci. USA 91: 4887-4891; Chance 1998, Ann. NY Acad. Sci. 838: 29-45), fiffuse optical tomography (Cheng et al., 1998 Optics Express 3: 118-123; Siegel et al. 1999, Optics Express 4: 287-298), intravital microscopy (Dellian et al., 2000, Br. J. Cancer 82: 1513-1518; Monsky et al. 1999 Cancer Res. 59: 4129-4135; Fukumura et al. 1998, cell 94: 715-725) and confocal imaging (Korlach et al. Proc. Natl. Acad. Sci. USA 96: 8461-8466; Rajadhyaksha et al. 1995, J. Invest. Dermatol. 104: 946-952; Gonzalez et al. 1999, J. Med. 30: 337-356), and others as will be appreciated by the skilled artisan.

In another embodiment, the methods of this invention are directed to the imaging of individual cells, a group of cells, a tissue, an organ or a combination thereof.

In one embodiment, imaging is accomplished with computed tomography, computed radiography, magnetic resonance imaging, fluorescence microscopy, angiography, arteriography, or a combination thereof. In one embodiment, a cell is contacted with a polymer of this invention, ex-vivo, and is subsequently implanted in a subject.

In one embodiment, the imaging methods of this invention are conducted on a subject. In another embodiment, the imaging methods are conducted on a sample taken from a subject. In one embodiment, the subject has or is suspected of having cancer.

In one embodiment, the imaging methods as described herein may comprise near infrared fluorescence imaging. In one embodiment, an advantages of such optical imaging methods may include the use of non-ionizing low energy radiation, high sensitivity with the possibility of detecting micron-sized objects, continuous data acquisition, and the development of potentially cost-effective equipment. Optical imaging can be carried out at different resolutions and depth penetrations. Fluorescence-mediated tomography (FMT) can three-dimensionally localize and quantify fluorescent probes in deep tissues at high sensitivity. Several NIR fluorochromes have recently been coupled to affinity molecules (Becker, A., et al. Nature Biotechnology, 19: 327-331, 2001; Folli, S., et al Cancer Research, 54: 2643-2649, 1994, and can be adapted to comprise the polymers of this invention, as will be appreciated by one skilled in the art.

In another embodiment, the polymers of this invention allow for the combination of different imaging modalities.

### Compositions

In one embodiment this invention provides a pharmaceutical composition comprising the polymers of this invention.

In one embodiment the composition further comprising a carrier, diluent, lubricant, flow-aid, or a mixture thereof. In one embodiment the composition is in the form of a pellet, a tablet, a capsule, a solution, a suspension, a dispersion, an emulsion, an elixir, a gel, an ointment, a cream, an I.V. solution or a suppository. In one embodiment the composition is in the form of a capsule. In one embodiment the composition is in a form suitable for oral, intravenous, intraarterial, intramuscular, intracranial, intranasal, subcutaneous, parenteral, transmucosal, transdermal, intratumoral or topical administration. In one embodiment the composition is a controlled release composition. In one embodiment the composition is an immediate release composition. In one embodiment the composition is a liquid dosage form. In one embodiment the composition is a solid dosage form. In one embodiment the composition further comprises an antineoplastic compound, an immunotherapeutic agent or a drug.

In another embodiment, this invention provides a composition comprising a polymer of this invention. In one embodiment this invention provides a pharmaceutical composition comprising the polymers of the present invention.

In one embodiment the composition further comprising a carrier, diluent, lubricant, flow-aid, or a mixture thereof. In one embodiment the composition is in the form of a pellet, a tablet, a capsule, a solution, a suspension, a dispersion, an emulsion, an elixir, a gel, an ointment, a cream, an I.V. solution or a suppository. In one embodiment the composition is in the form of a capsule.

Pharmaceutical compositions of this invention for parenteral injection comprise pharmaceutically acceptable sterile aqueous or nonaqueous solutions, dispersions, suspensions, or emulsions as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

In one embodiment the composition is in a form suitable for oral, intravenous, intraarterial, intramuscular, intracranial, intranasal, subcutaneous, parenteral, transmucosal, transdermal, rectally, intracisternally, intravaginally, intraperitoneally, topically (as by powders, ointments, or drops), bucally, or as an oral or nasal spray. The term “parenteral” administration as used herein refers to modes of administration which include intravenous, intramuscular, intraperitoneal, intrathecally, intrasternal, subcutaneous and intraarticular injection and infusion.

In one embodiment the composition can be administered to humans and other animals. In one embodiment the composition is a controlled release composition. In one embodiment the composition is an immediate release composition. In one embodiment the composition is a liquid dosage form. In one embodiment the composition is a solid dosage form. In one embodiment the composition further comprising an antineoplastic compound, an immunotherapeutic agent or a drug. In one embodiment, the compositions of this invention, which comprise a polymer of this invention is biocompatible, and in another embodiment, may comprise pharmaceutically acceptable carriers or excipients, such as disclosed in Remington's Pharmaceutical Sciences, Mack Publishing Company, Easton, Pa., USA, 1985. The polymers, of this invention may be used in the treatment or diagnosis of certain conditions such as in tagging, detecting or removing cancer cells for example from a sample or tissue. These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drag then depends upon its rate of dissolution which, in turn, may depend upon crystal size and crystalline form. Alternatively, delayed absorption of a parenterally administered drug form is accomplished by dissolving or suspending the drag in an oil vehicle.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions for rectal or vaginal administration are, in one embodiment, suppositories which can be prepared by mixing the compounds of this invention with suitable non-irritating excipients or carriers such as cocoa butter, polyethylene glycol, or a suppository wax which are solid at room temperature but liquid at body temperature and therefore melt in the rectum or vaginal cavity and release the active compound.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the polymer compound of the present invention, stabilizers, preservatives, excipients, and the like. In one embodiment, the lipids may be natural or synthetic phospholipids or a combination thereof.

Methods to form liposomes are known in the art. See, for example, Prescott, Ed., Methods in Cell Biology, Volume XIV, Academic Press, New York, N.Y. (1976), p. 33 et seq.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend as upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved.

The pharmaceutical compositions of the present invention can be used in both veterinary medicine and human therapy. The magnitude of a prophylactic or therapeutic dose of the pharmaceutical composition of the invention will vary with the severity of the condition to be treated and the route of administration. The dose, and perhaps the dose frequency, will also vary according to the age, body weight, and response of the individual patient.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

This invention provides a polymer, which in one embodiment, is water soluble. In one embodiment, water soluble polymers allow for the polymers to be delivered through the blood stream. The polymers of this invention, in some embodiments, offer a number of advantages as delivery systems, as compared to other such systems described in the art, as a result of the unique chemical structure of the polymers of this invention.

The polymers of this invention may assume any structural configuration, which will be a function of, in some embodiments, the chemical makeup of the polymers, and the environment to which the polymer is exposed. In some embodiments, the polymers of this invention may assume a particle configuration.

In other embodiments, the polymers of this invention may comprise a targeting agent. In one embodiment, the targeting agent serves for diagnostic and/or imaging purposes, where an agent is delivered to a particular site, where verification of delivery is desired. In another embodiment, the targeting agent serves to provide a sensitive means of detection of a particular molecule at a particular site, for example, the targeting agent directs a polymer of this invention to a tissue which expresses a preneoplastic marker, or a cancer associated receptor or molecule, wherein the molecule which is being detected is available in low concentration, and in some embodiments, is not detectable by existing methods in the art.

In some embodiments, the targeting agent may be coupled to a free HPMA unit at an end of a base polymer chain.

In some embodiments, through the use of various chain lengths, linkers, side chains, and side chain terminal groups, great flexibility in polymer chemical composition, size, structure, and function can be obtained. In some embodiments, such polymers may be constructed via multiple-step reaction pathways that involve synthesis of a suitable monomer with a protected functional group prior to the polymerization step, followed by deprotection. In other embodiments, the synthesis may be carried out with a chemical/enzymatic/chemo-enzymatic approach as exemplified and described further herein.

Synthesis of the polymer precursors or of the polymers of this invention may be carried out in a number of representative suitable solvents including anhydrous polar aprotic solvents such as acetonitrile, tetrahydrofuran, dioxane, or the like, halogenated solvents such as chloroform, or the like. In some embodiments, synthesis is conducted as exemplified herein, or as a variation thereof, as will be appreciated by the skilled artisan. Synthesis of the monomeric units of the polymers and their linkage to other monomeric units are understood to reflect the choice of monomeric unit and can be accomplished by routine methodology known in the art.

In another embodiment, the polymers are synthesized enzymatically. In one embodiment, the enzymes used to synthesize the polymers of this invention comprise lipases, such as, for example Candida antarctica lipase, or in another embodiment, lipase A, or in another embodiment, lipase B. In another embodiment, the enzyme may comprise an esterase, or in another embodiment, a protease, such as, for example papain or chymotrypsin. In one embodiment, molecular weight of the hydrophilic units is chosen such that its ability to affect polymerization is considered. In one embodiment, the polymer is functionalized with for example, an alkyl group of varying chain length, comprising a polar functionality at the end of the chain.

Polymers obtained by methods as described herein can be characterized by methods well known in the art. For example, the molecular weight and molecular weight distributions can be determined by gel permeation chromatography (GPC), matrix assisted laser desorption ionization (MALDI), and static or dynamic light scattering. Physical and thermal properties of the polymer products can be evaluated by thermal gravemetric analysis (TGA), differential scanning calorimetry (DSC), or surface tensiometer; the chemical structures of the polymers can be determined by, e.g., NMR (1H, 13C NMR, 1H-1H correlation, or 1H-13C correlation), IR, UV, Gas Chromatography-Electron Impact Mass Spectroscopy (GC-EIMS), EIMS, or Liquid Chromatography Mass Spectroscopy (LCMS).

In some embodiments this invention is related to the imaging an inflammatory condition in a subject, the method comprising administering a polymer of this invention, or a composition of this invention to said subject

In one embodiment this invention provides a method of imaging a disease associated with neovascularization in a subject, said method comprising administering a polymer of this invention, or a composition of this invention to said subject.

In one embodiment, this invention provides a method of imaging a cancer or cancerous tissue in a subject, the method comprising the step of contacting a cancer or cancerous tissue with a polymer of this invention, or a composition of this invention.

In one embodiment, the polymer binds to receptors on the neoplastic cells via its targeting moiety.

In one embodiment, the polymer is administered intra-tumorally.

In one embodiment the polymer comprises a spacer comprising a cleavable moiety. In one embodiment the cleavable moiety is a tetra-peptide. In one embodiment the tetra-peptide is (Gly-Phe-Leu-Gly). In one embodiment the cleavage is induced chemically. In one embodiment the cleavage is induced after the polymer binds the neoplastic cell. In one embodiment the cleavage is induced by cysteine peptidases. In one embodiment the cysteine peptidase is cathepsin B. In one embodiment the source of said cathepsin B is the lysosomal compartments of tumor cells.

In one embodiment this invention provides a method of diagnosing cancer in a subject, wherein the method comprising contacting a polymer of the present invention to a neoplastic cell or vasculature associated with a neoplastic cell in the subject. In one embodiment the diagnosis comprises the detection of the tag moiety on the polymer. In one embodiment the tag moiety is 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide. In one embodiment the detection of the tag moiety is an optical detection.

In one embodiment, the term “administering” refers to bringing a subject in contact with the indicated agent. In another embodiment, administration is accomplished in vitro, i.e. in a test tube. In another embodiment, administration is accomplished in vivo, i.e. in cells or tissues of a living organism. Each possibility represents a separate embodiment of the present invention.

In one embodiment cancers are classified by the type of cell that resembles the tumor and, therefore, the tissue presumed to be the origin of the tumor. In one embodiment the cancer type is carcinoma, in which Malignant tumors are derived from epithelial cells. In one embodiment carcinoma represents the most common cancers, including the common forms of breast, prostate, lung and colon cancer. In another embodiment the cancer type is sarcoma. In one embodiment this type of cancer comprises malignant tumors derived from connective tissue, or mesenchymal cells. In another embodiment the cancer type is lymphoma or leukemia. In one embodiment this cancer type comprises malignancies derived from hematopoietic (blood-forming) cells. In another embodiment the cancer type is in the form of a germ cell tumor. In one embodiment such tumor is derived from totipotent cells. In another embodiment, the tumor is a blastic tumor. In one embodiment this is a usually malignant tumor which resembles an immature or embryonic tissue.

In some embodiments, the compounds/compositions and methods of this invention are useful in the diagnosis of any vascularized tumor, for example, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas).

In some embodiments, the compounds/compositions and methods are useful in diagnosing other diseases associated with neovascularization, such as, but not limited to inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells

Other angiogenesis-associated diseases or disorders which can be diagnosed with the compounds/compositions or by the methods encompassed by the present invention include, but are not limited to, osteoarthritis, lupus, systemic lupus erythematosis, polyarteritis, artery occlusion, vein occlusion, carotid obstructive disease, sickle cell anemia, pseudoxanthoma elasticum, Paget's disease, lyme's disease, Best's disease, Eale's disease, Stargardt's disease, toxoplasmosis, phylectenulosis, lipid degeneration, chronic inflammation, atherosclerosis, hereditary diseases, such as Osler-Weber-Rendu disease.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

The following examples are presented in order to more fully illustrate some embodiments of the invention. They should, in no way be construed, however, as limiting the scope of the invention.

### Example 1

### Synthesis of Targetable Polymer Conjugates

Synthesis of IR-783 Dye with a Free Carboxylic Acid Group (IR-783-S-Ph-COOH)

IR-783-S-Ph-COOH was synthesized based on a previously described procedure (Wang et al., Bioconjugate Chem., Vol. 18, No. 2, 2007) (see scheme 1 below). Briefly, IR-783 was conjugated with 4-mercaptobenzoic acid in DMF in the presence of DIPEA at 1:1:1 molar ratio. The mixture was stirred over night. The solvent was evaporated and the product was purified by silica gel column, mobile phase ethylacetate:methanol (1:1) and analyzed by MALDI. Yield: 92%.

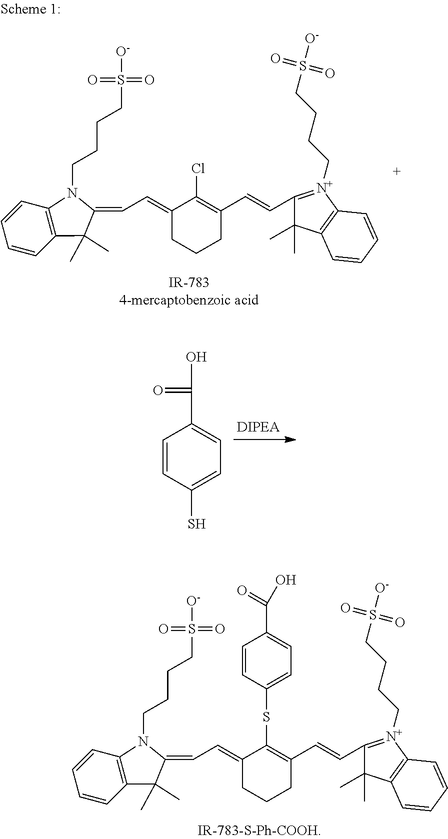 [SMILES: CC1(C)C(/C=C/C2=C(Cl)C(=C/C=C3\N(CCCCS(=O)(=O)[O-])c4ccccc4C3(C)C)/CCC2)=[N+](CCCCS(=O)(=O)[O-])c2ccccc21 ; O=C(O)c1ccc(S)cc1 ; CC1(C)C(/C=C/C2=C(Sc3ccc(C(=O)O)cc3)C(=C/C=C3\N(CCCCS(=O)(=O)[O-])c4ccccc4C3(C)C)/CCC2)=[N+](CCCCS(=O)(=O)[O-])c2ccccc21]

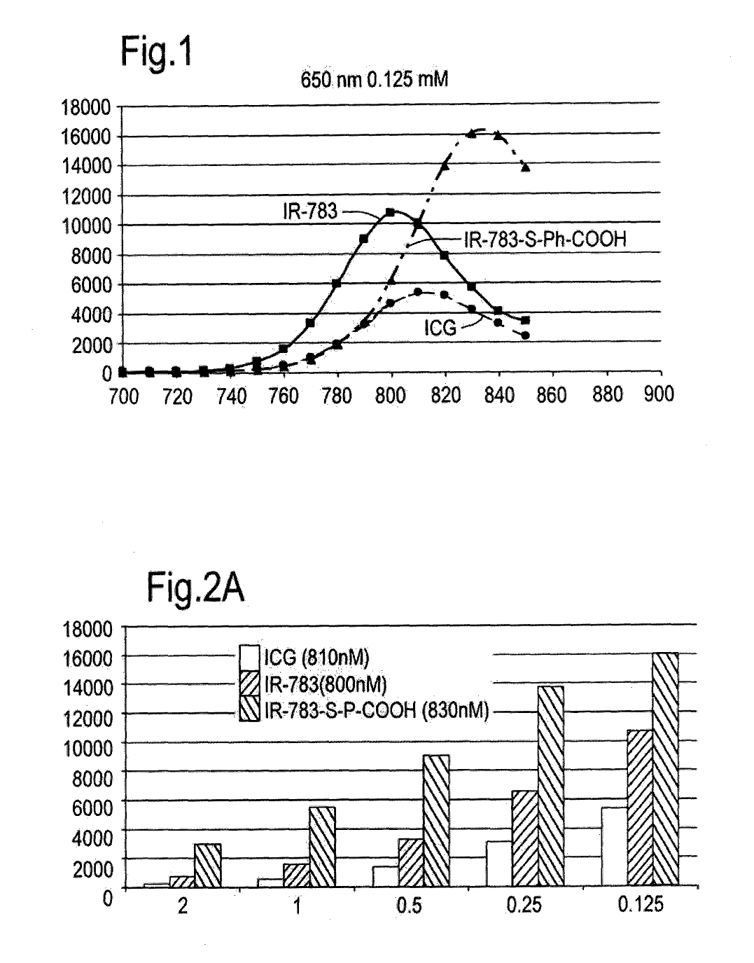

FIG. 1 depicts the emission spectrum for NIR Dyes (ICG, IR-783, and 783-S-Ph-COOH) following excitation at 650 nm (A) and 690 nm (B).

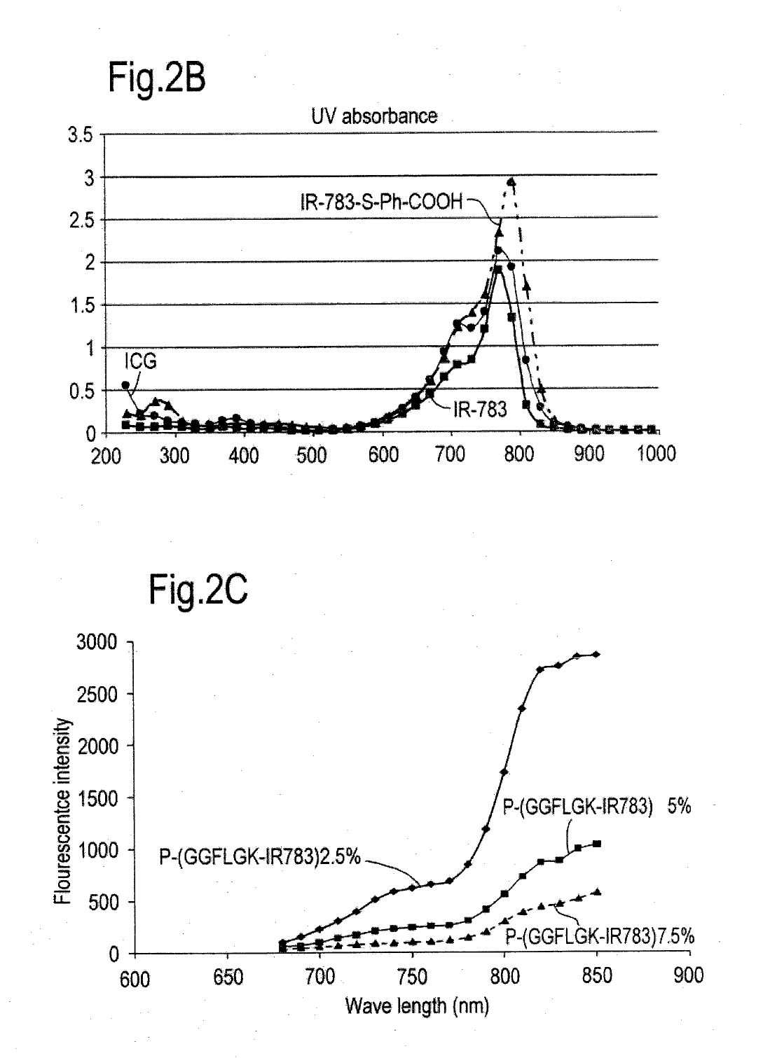

Fluorescence intensity of the NIR dyes was evaluated as well, following excitation at 650 nm (FIG. 2A). The intensity was measured at the maximal emission wavelength for each dye: 800 nm (IR-783), 810 nm (ICG) and 830 nm (IR-783-S-Ph-COOH). Absorption spectrum of the NIR dyes is shown in FIG. 2B.

### Synthesis of Polymer Precursor for IR-783-S-Ph-COOH Attachment:

An HPMA copolymer precursor incorporating the cathepsin B cleavable spacer GFLGK for attachment to IR-783-S-Ph-COOH attachment (designated as P-(GFLGK)-Boc, where P represents the HPMA copolymer backbone) was synthesized by random radical precipitation copolymerization in a sealed vial in acetone/DMSO mixture at 50° C. for 24 hr using AIBN as the initiator (see Scheme 2). The feed molar percentage of the monomers was 85:15 for HPMA and MA-GFLGK-(Boc)-COOH, respectively. The ratio of monomers to initiator and solvent was 12.5:0.6:86.9 wt %, respectively. The content of the monomers in the copolymer was calculated by H¹-NMR.

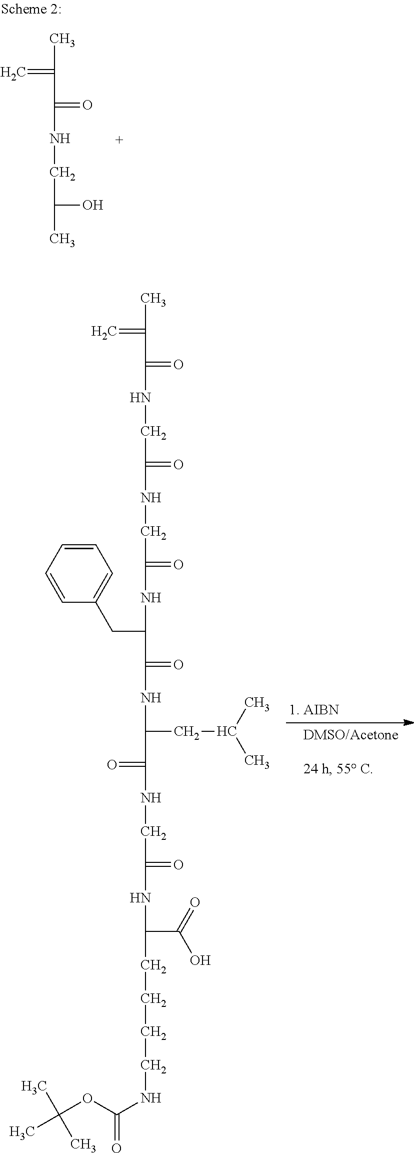

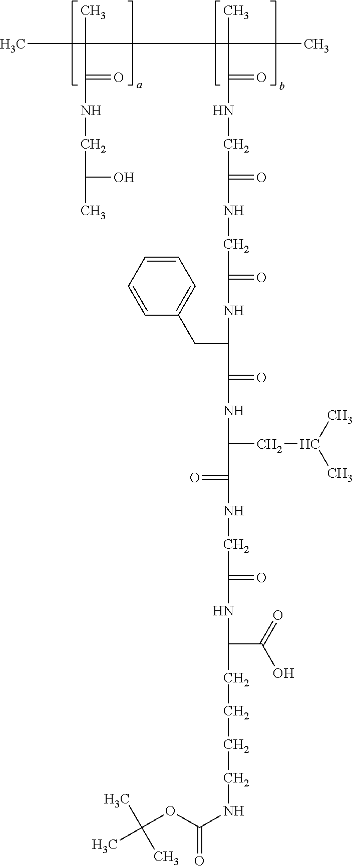

### Synthesis of IR-783-S-Ph-COOH Containing Copolymer:

The HPMA precursor copolymer (P-GFLGK-Boc) was dissolved in 100% TFA for 8 min to remove the Boc protecting group to yield P-GFLGK-NH₂. The solution was concentrated by evaporation, and the polymer precipitated in cold ether, and dried.

A molar ratio of 1:5 between GFLGK-NH₂ group and IR-783-S-PH-COOH was used in the reaction mixture for coupling the NIR dye. IR-783-S Ph-COOH and the coupling reagents HBTU and DIPEA were first dissolved in DMF and kept in the molar ration of (1:1:6). After 3 minutes the polymer P-GFLGK was added and the reaction mixture was stirred overnight at room temperature. The NIR conjugated copolymer was then precipitated in acetone:ether (1:1), dried, and purified on Sephadex (LH-20) column (using DDW as eluent).

The synthesis outline can be seen in Scheme 3:

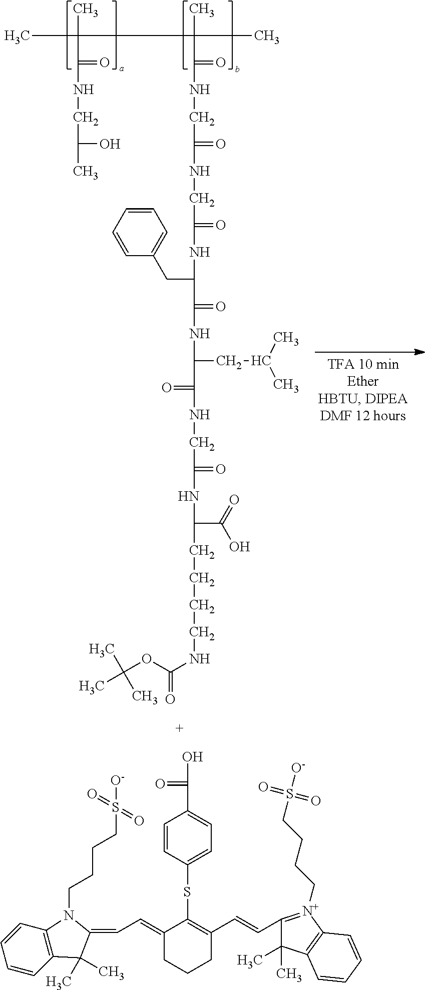

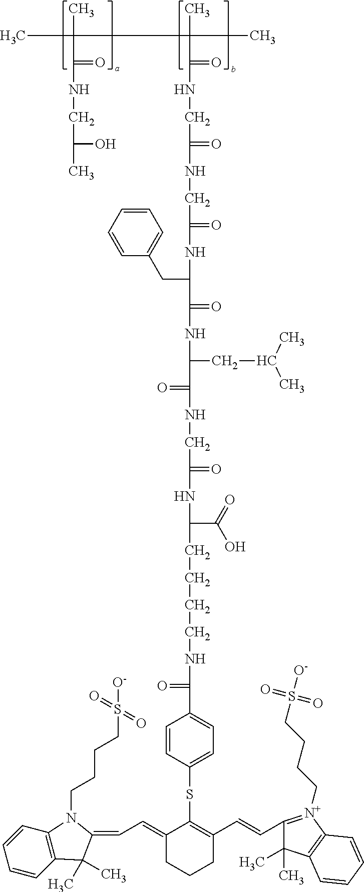

Copolymers as characterized in Table I were synthesized based on the methods described hereinabove.

TABLE 1
Characterization of NIR labeled copolymers.

| | | | | | Number |
|---|---|---|---|---|---|
| | Type of linker | | | | fluorescent |
| | for IR-783 | Approx. | | % Mol IR783- | molecules per |
| Copolymer code | attachment | Mw (Da)^{a} | P_{I}^{a} | S—Ph—COOH^{b} | polymer chain |
| P-(GGFLGK-IR783)_{2.5%} | Degradable | 63,000 | 2.4 | 1.8 | 7 |
| P-(GGFLGK-IR783)_{5%} | Degradable | 86,500 | 2.9 | 4 | 19 |
| P-(GGFLGK-IR783)_{7.5%} | Degradable | 56,000 | 2.3 | 5 | 15 |
| P-(APMA-IR783)_{2.5%} | Non-degradable | 120,000 | 3.3 | 1.5 | 12 |
| P-(APMA-IR783)_{7.5%} | Non-degradable | 144,000 | 3.8 | 5 | 36 |

^{a}The weight average molecular weights of copolymers were estimated by size-exclusion chromatography.
^{b}The content of NIR dye was determined by ¹H-NMR and spectrophotometrically.
^{c}The contents of targeting moieties were estimated by ¹H-NMR

HPMA conjugates of various IR-783-S-Ph-COOH loadings were dissolved in DDW and their fluorescence intensity (Ex: 690 nm, Em: 820 nm). The results indicate that polymer with 2.5 mol % of IR-783-S-Ph-COOH loading dye (P-(GGFLGK-IR783)_{2.5}%) (7 dye molecules per polymer chain) exhibit the highest fluorescence intensity at λ=820 nm, when compared to the copolymers bearing 5% (P-(GGFLGK-IR783)₅%) and 7.5% of IR-783-S-Ph-COOH (P-(GGFLGK-IR783)_{7.5}%) (with an about 19 and 15 dye molecules per polymer chain, respectively) (FIG. 2C). These observations confirm the decrease in fluorescence intensity with increasing the loading of IR-783-S-Ph-COOH on p-HPMA copolymer due to the quenching of fluorescent signal.

### Results

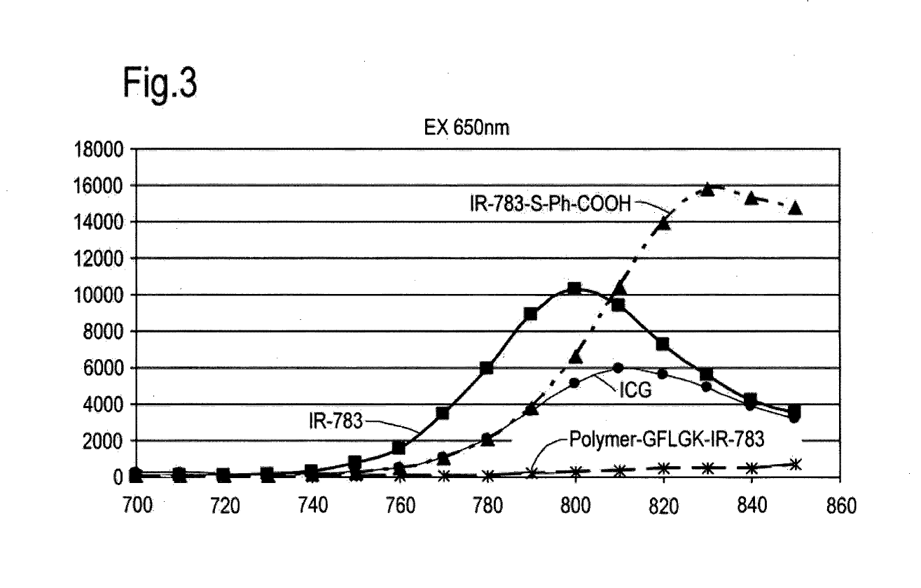

The effect of NIR813 loading on p-HPMA-NIR813 quenching efficiency is shown in FIG. 3 and a complete quenching was achieved when the loading level of IR-783 on P—HPMA-IR-783 was 15%.

### Example 2

### Target Specific Activation of Fluorescence

### Assaying Cathepsin Degradation of the Linker:

0.5 mg polymer was dissolved in 1 ml sodium acetate buffer (pH=5.5). Cathepsin B (1.5 U) was added to the solution and incubate for 24 hours at 370 C. The fluorescence intensity (excitation 650 nm & 690 nm, emission range 820 nm) was measured every 30 minutes.

### Results

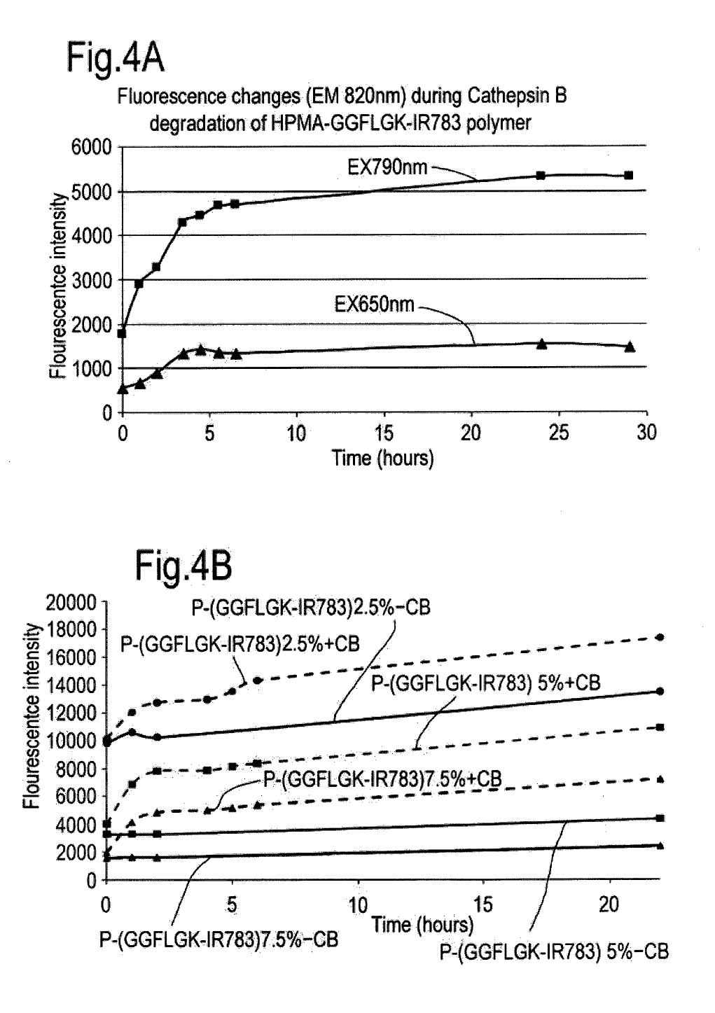

The effects of IR-783-S-Ph-COOH loading on CB mediated fluorescence activation was tested. Fluorescence intensity clearly increased as a consequence of Cathepsin degradation (FIG. 4A). We found that the extent of recovered fluorescence intensity following CB degradation has increased with increasing the incubation time. HPMA copolymer containing 5% (P-(GGFLGK-IR783)₅%) and 7.5% (P-(GGFLGK-IR783)_{7.5}%) IR-783-S-Ph-COOH dye, exhibited 3.6-fold and 4.9-fold increase in the intensity after 22 h of incubation respectively, while the copolymer bearing 2.5% IR-783-S-Ph-COOH loading (P-(GGFLGK-IR783)_{2.5}%) showed only 2-fold increase in signal intensity over time, which may be attributed to lack of efficient quenching to begin with (FIG. 4B).

### Example 3

### In Vivo Application of Targeted Copolymers

A polymeric imaging probe that can actively and specifically recognize in vivo underglycosylated mucin-1 antigen (uMUC-1) antigen in an animal model of human CRC was designed and synthesized. uMUC-1 is one of the early hallmarks of tumorigenesis and is overexpressed and underglycosylated on almost all human epithelial cell adenocarcinomas, including colon cancer.

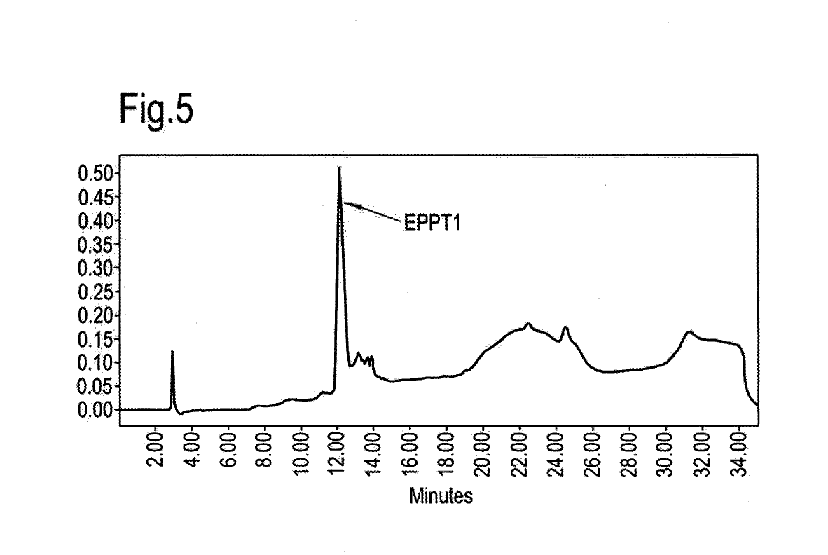

EPPT1 was synthesized with a protected Lys residue, of primary sequence: YCAREPPTRTFAYWG (SEQ ID NO: 10)-K-Boc using Fmoc solid phase peptide synthesis (SPPS) on a Rink Amide MBHA resin. The Fmoc protecting group was removed from the resin by exposure twice to 20% piperidine for 8 min. Each amino acid (0.1 mmol) was dissolved in DMF containing HBTU (0.1 mmol/ml) and DIPEA (0.1 ml), stirred for 3 min and then added to the reaction syringe. Coupling reaction was performed for 45 min after which the resin was washed with DMF and reacted twice with 20% piperidine for 8 min. The peptides were cleaved from the resin using mixture of TFA:TIS:H2O (95:2.5:2.5) for 2 h. The peptides were precipitated in cold ether, centrifuged, dried and characterized using HPLC and MALDI-TOF. The purity of the product was estimated by reverse phase analytical HPLC in a C18 column using linear water (Buffer A) and acetonitrile (Buffer B) gradient. (Buffer A: 99% water, 1% acetonitrile, 0.1% TFA; Buffer B: 90% acetonitrile, 10% water, 0.07% TFA) (FIG. 5).

The EPPT1 peptide is then coupled to FITC or IR-783-labeled copolymer precursors containing reactive ONp ester groups (P-(GG-ONp)-FITC and P-(GG-ONp)-(GGFLGK-Boc), respectively) via aminolysis, as described hereinabove. The IR783-S-Ph-COOH is then coupled to the P-(EPPT1)-(GGFLGK-Boc) following the removal of the Boc protecting group by TFA.

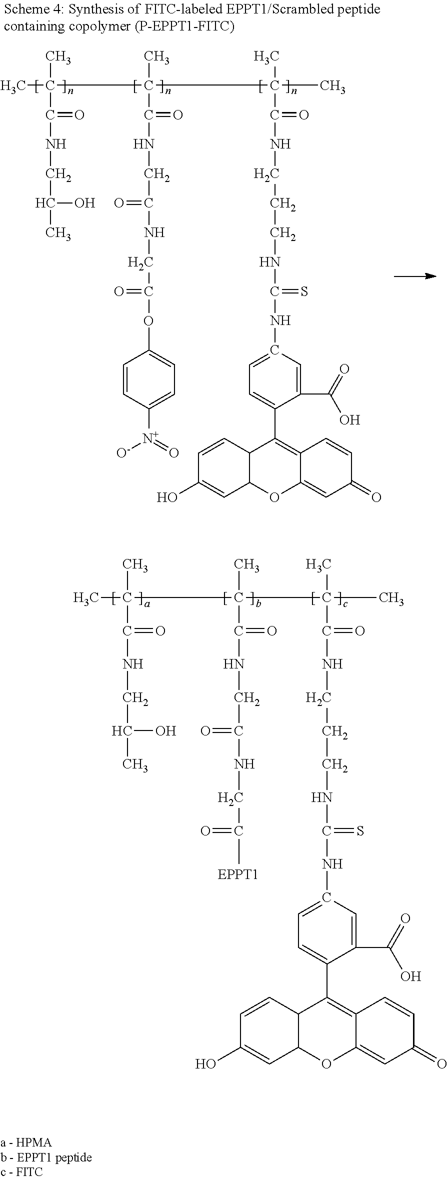

### Example 4

### In Vivo Application of Targeted Copolymers

Three types of mouse models were employed to test the ability of the probes to detect solid tumors in the GI tract.

Nu/nu athymic mice were injected orthotopically into the descending colon of female with SW-480 cells. After tumors reached ˜0.5 cm in diameter, mice were injected i.v. with 2 mg of IR-783 bearing polymeric probe. The results in the orthotopically implanted tumors confirm the accumulation of both P-(GGFLGK (SEQ ID NO: 11)-IR783)_{2.5}% and P-(GGFLGK (SEQ ID NO: 11)-IR783)_{7.5}% polymeric probes in tumor area about 4 h post injection and retention at the tumor site for at least 48 h.

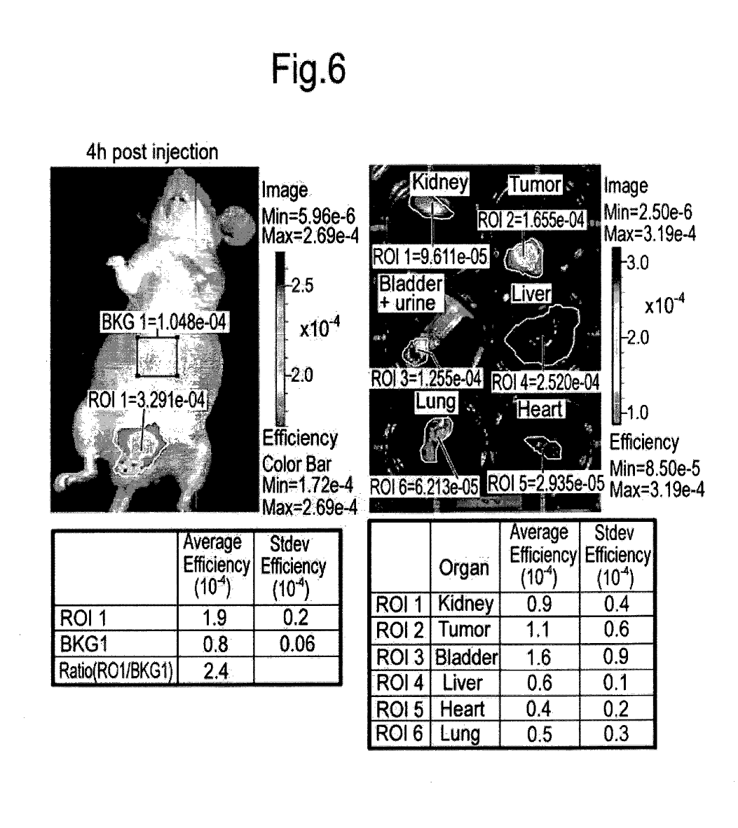

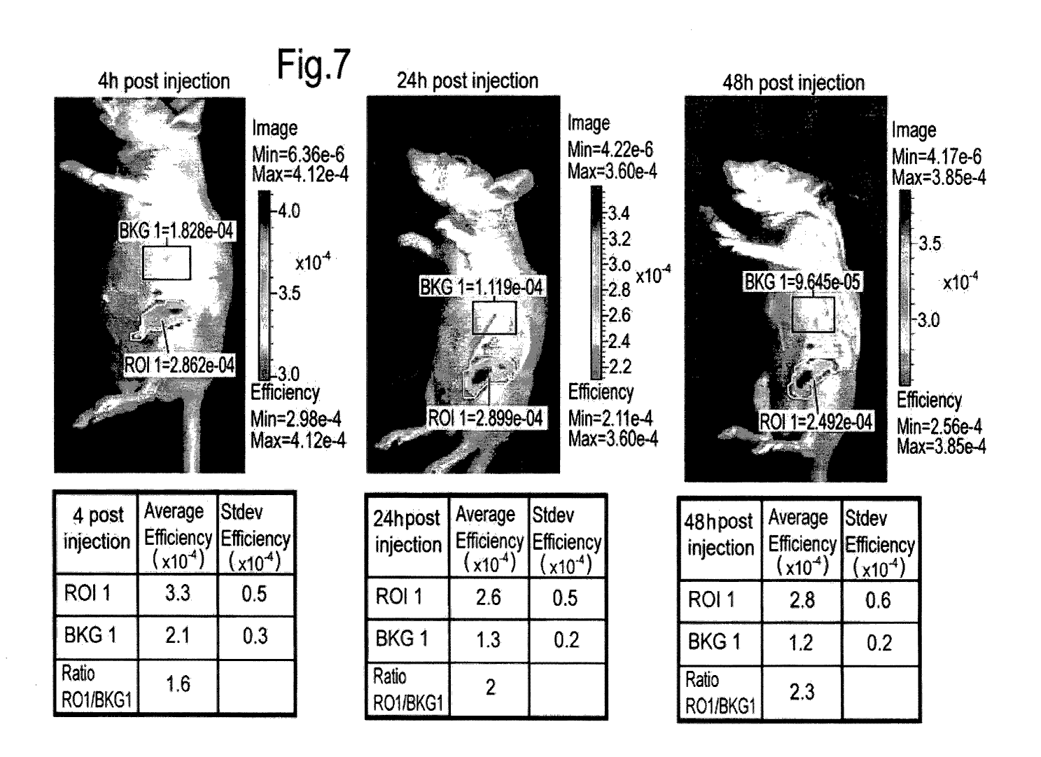

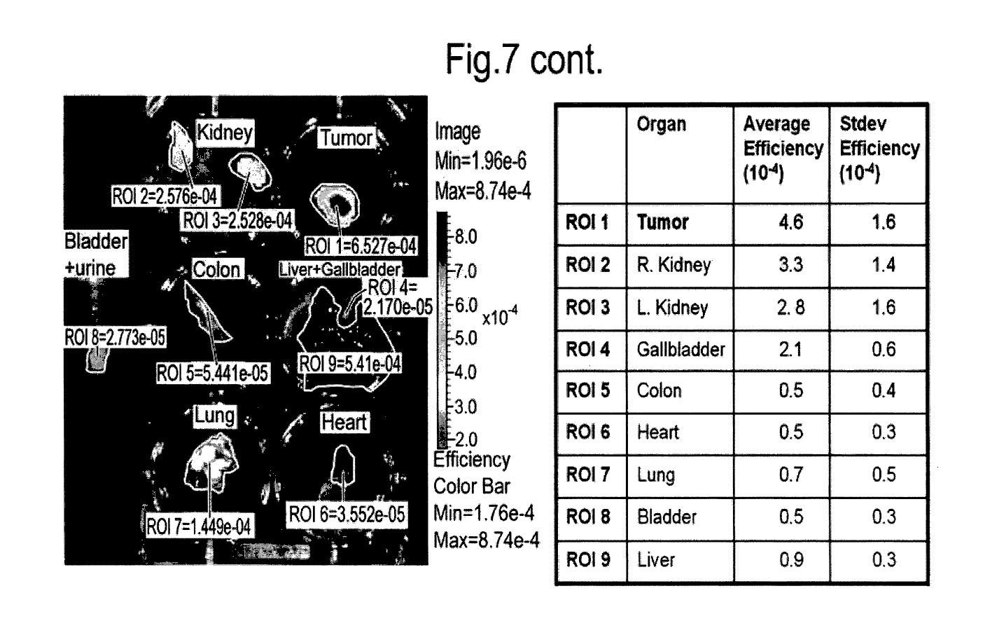

Biodistribution analysis indicated the presence of the probe in the tumor, kidneys, galbladder and the urine. The T/B ratio following whole body imaging (WBI) was 2.4 in mice treated with P-(GGFLGK (SEQ ID NO: 11)-IR783)_{7.5}%, 4 h post injection (FIG. 6). Biodistribution analysis of P-(GGFLGK(SEQ ID NO: 11)-IR783)_{2.5}% polymeric probe in the mice sacrificed 48 h post injection showed a significant accumulation in the tumor, kidneys and gallbladder. The calculated ratio of the average fluorescence efficiency between colon and tumor tissue was ˜9 (FIG. 7).

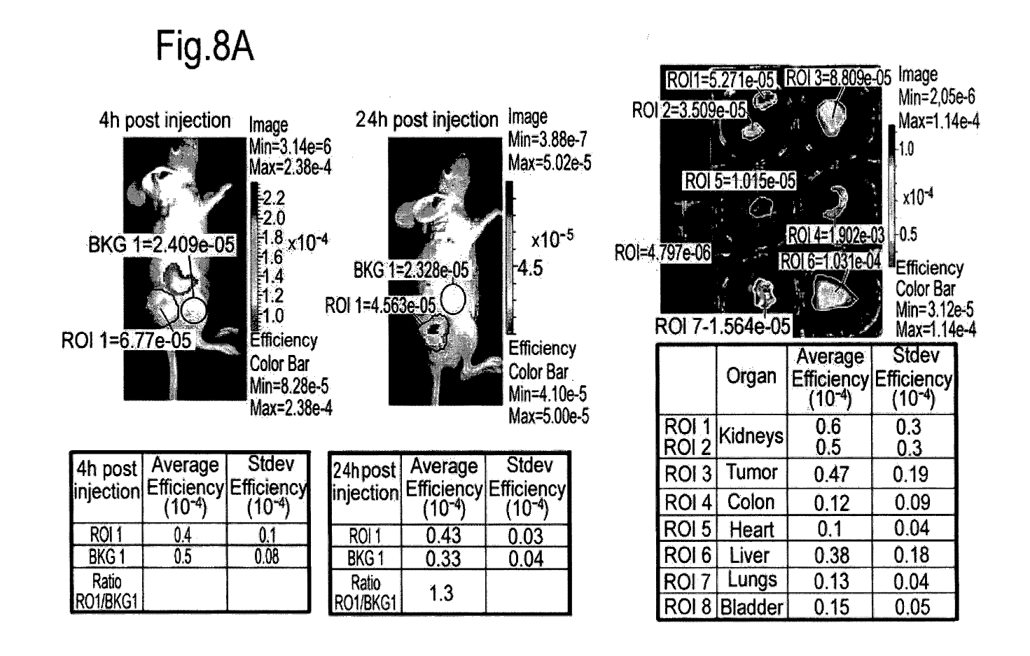

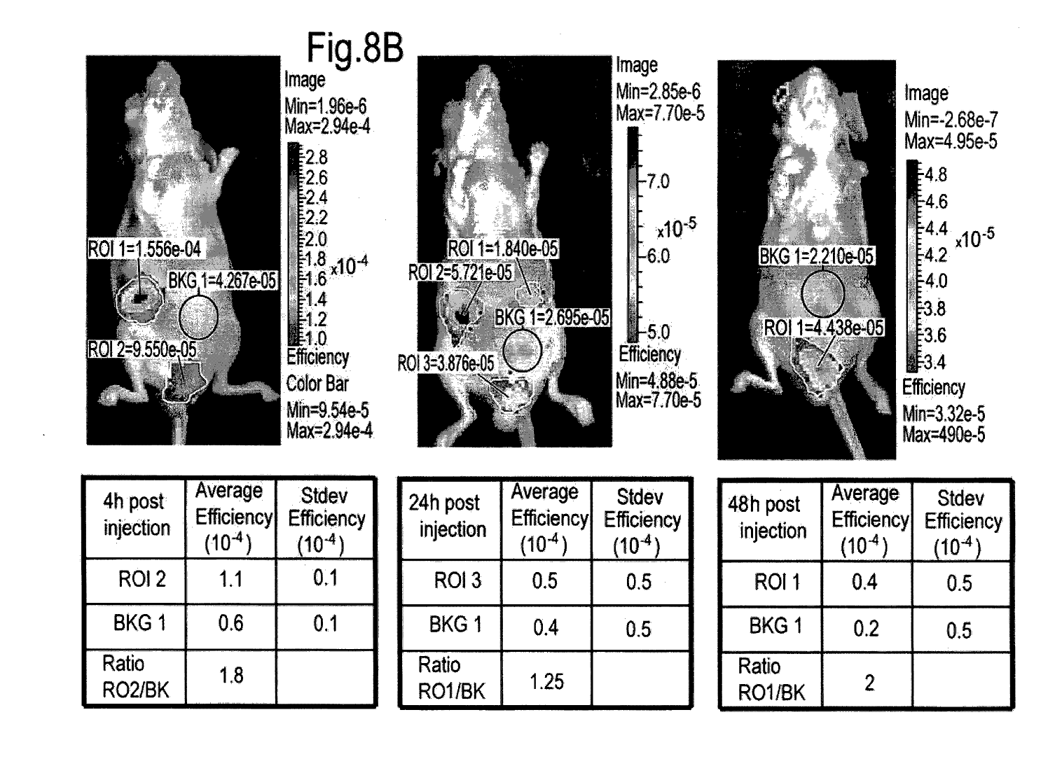

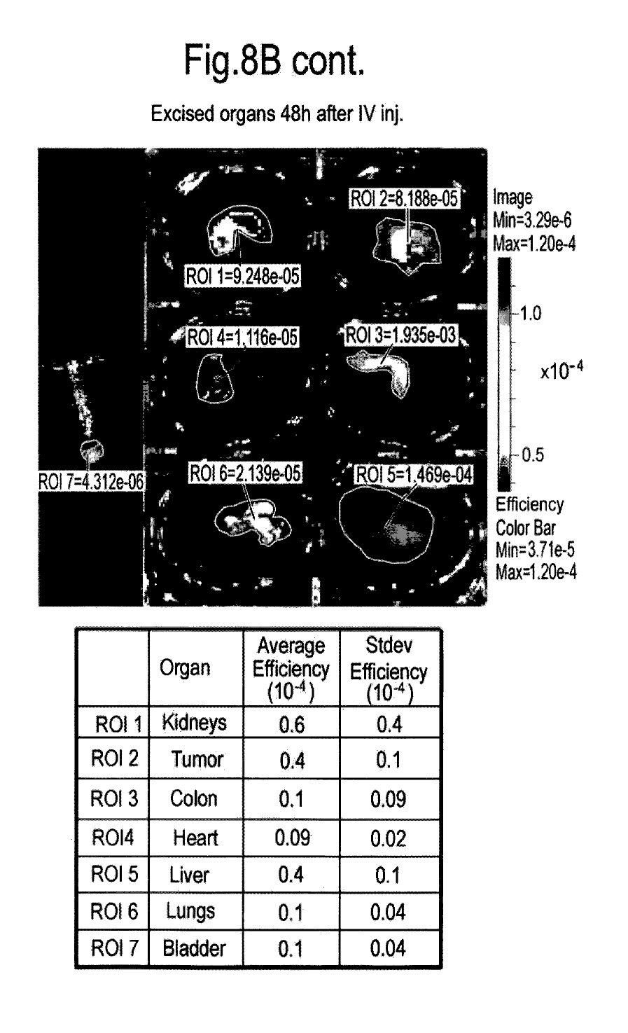

The ability of the probe to detect solid tumors in female mice bearing rectally tumors injected with SW-480 cells was tested. The mice were injected with 0.2 mg/200 μl of P-(GGFLGK (SEQ ID NO: 11)-IR783)2.5% and the whole body was imaged 4 and 24 h post injection. The T/B ratio was not significantly different in whole body imaging 4 and 24 h post injection. However images from excised organs taken 24 h post injection indicated a 4-fold increase in the average fluorescence efficiency between colon and tumor tissue (FIG. 8A). When mice were injected with P-(GGFLGK (SEQ ID NO: 11)-IR783)7.5%, a slight increase in the tumor accumulation with time was noted. Images from excised tumor harvested 48 h post injection showed a ratio of ˜4 in the average fluorescence efficiency between colon and tumor tissue (FIG. 8b).

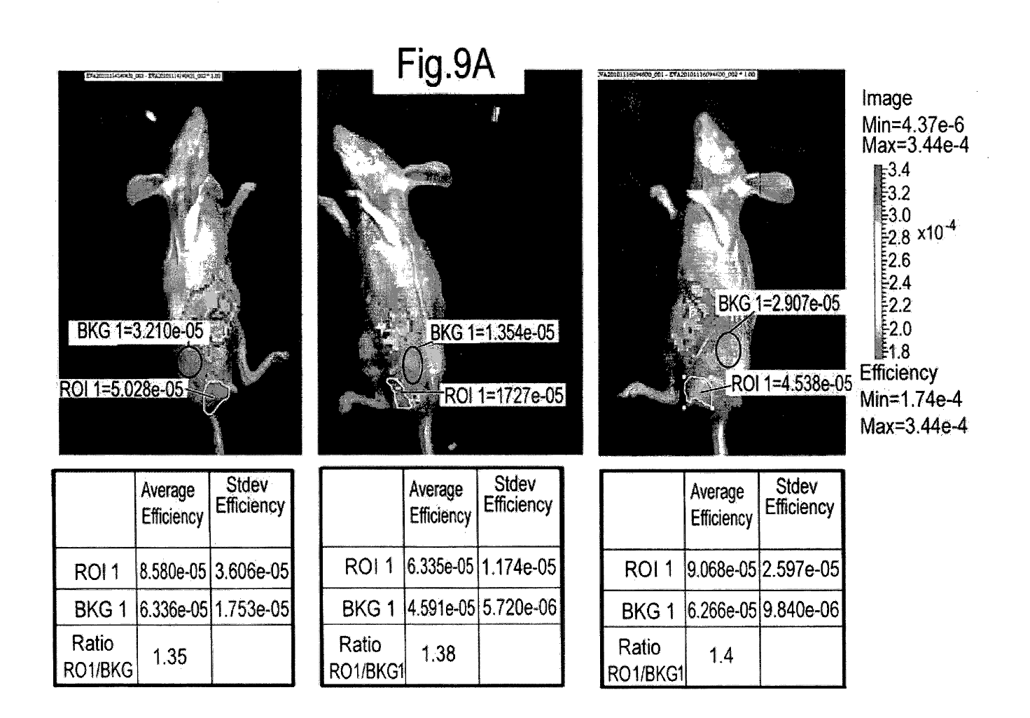

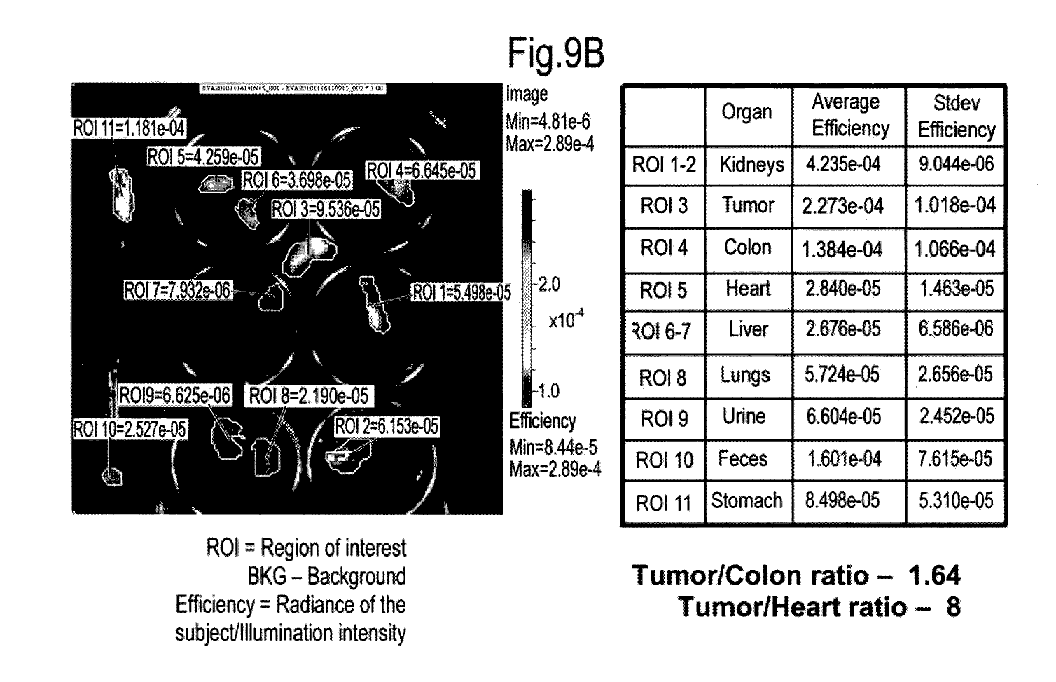

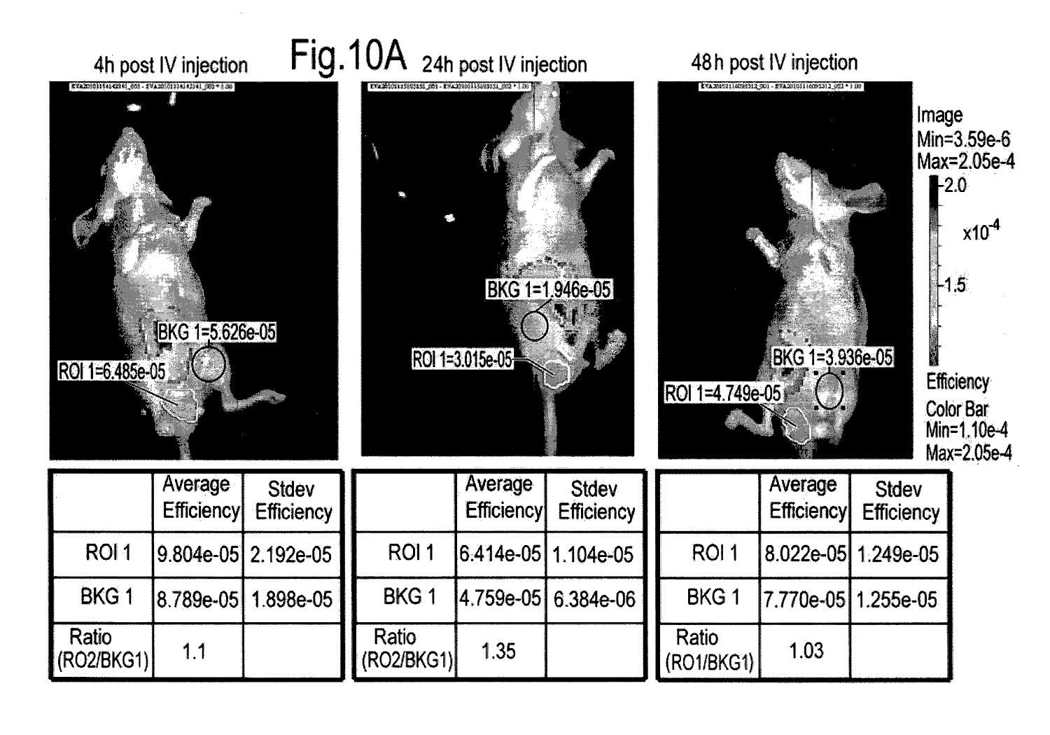

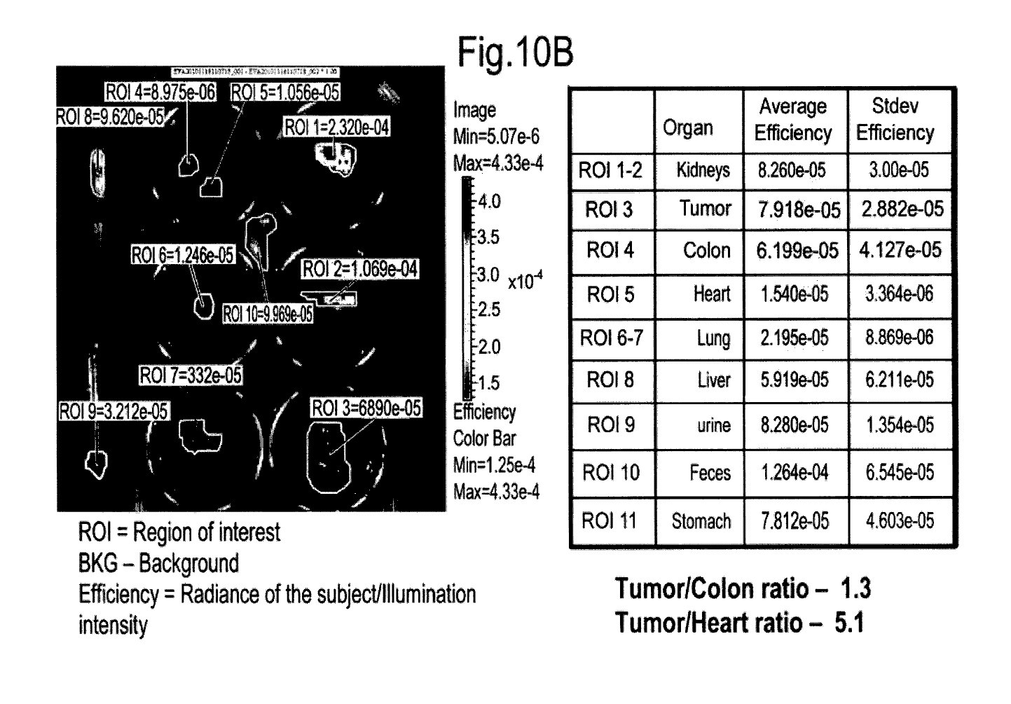

The ability of the probe to detect solid tumors in female mice bearing rectal tumors introduced via injection with HT-29 cells was also evaluated. After tumors reached ˜0.5 cm in diameter, mice were injected i.v. with 2 mg of IR-783 bearing polymeric probe. Mice were kept in metabolic cages throughout the experiment (48 h). The mice were injected with 1 mg/200 μl of P-(GGFLGK (SEQ ID NO: 11)-IR783)7.5% (CB cleavable linker) and the whole body was imaged 4, 24 and 48 h post injection. In accordance with model 2 (SW-480 cells injected rectally), the T/B ratio in HT-29 rectal model was not significantly different in whole body imaging 4 and 24 h post injection (FIG. 9a, FIG. 10a) indicating no increase in the tumor accumulation with time. In images from excised organs taken 24 h post injection indicated only 1.5-fold increase in the average fluorescence efficiency between colon and tumor tissue (T/C) (1.64 and 1.3-fold of increase, FIG. 9b and FIG. 10b, respectively), due to the high background fluorescence along the gastrointestinal tract (stomach, colon and fetal), even though treated in metabolic cages. Once the tumor to heart ratio (T/H) was measured, an increased of about 8-10-fold was calculated. When mice were injected with the polymeric probe with non-cleavable linker P-(AP-IR783)7.5%, and the whole body was imaged 4 post injection, the T/B ratio was 1.3. Unfortunately, the mice did not survive the treatment.

The imaging probes used hereinabove were indeed able to detect solid tumors after IV administration. Macromolecular imaging probes were shown to passively accumulate in solid tumor due to EPR effect as soon as 4 hours post injection. This was true for all the different copolymers; P-GGFLGK-IR783 bearing 2.5 and 7.5 molar percentage of IR-783-S-Ph-COOH dye, without the use of a targeting ligand. When whole body imaging was conducted, no significant differences in the T/B ratio were found following the treatment with the different copolymers at various doses (in all cases, the fold of increase was ˜0.2). In addition, no increase in the T/B ratio following whole body imaging was detected when increasing the incubation time from 4 to up to 48 h incubation, in all tested probes. (T/B ratio was ˜2). However, the average fluorescence efficiency was increased with time in excised organs, and the tumor to colon ratio was about ˜4-10, meaning 2-5-fold higher than what was observed in whole body imaging. This can be explained by the lower sensitivity of the IVIS Lumina system following whole body imaging procedure relative to the excised organs. It is very important to keep in mind that all the calculations of T/B are performed relative to an areas that were selected as region of interest (ROI) (=T) or as background (=B). In whole body imaging it is impossible to determine the exact location of the tumor or the different organs, and thus ratios calculated in the whole body imaging are less accurate when compared with the calculation based on excised organs (tumor to colon ratio). A 4-10 fold of increase in the average fluorescence intensity from excised organs might be sufficient to guide selective removal of polyps during colonoscopic procedures and aid the screening procedure when using the Pillcam® video camera technology.

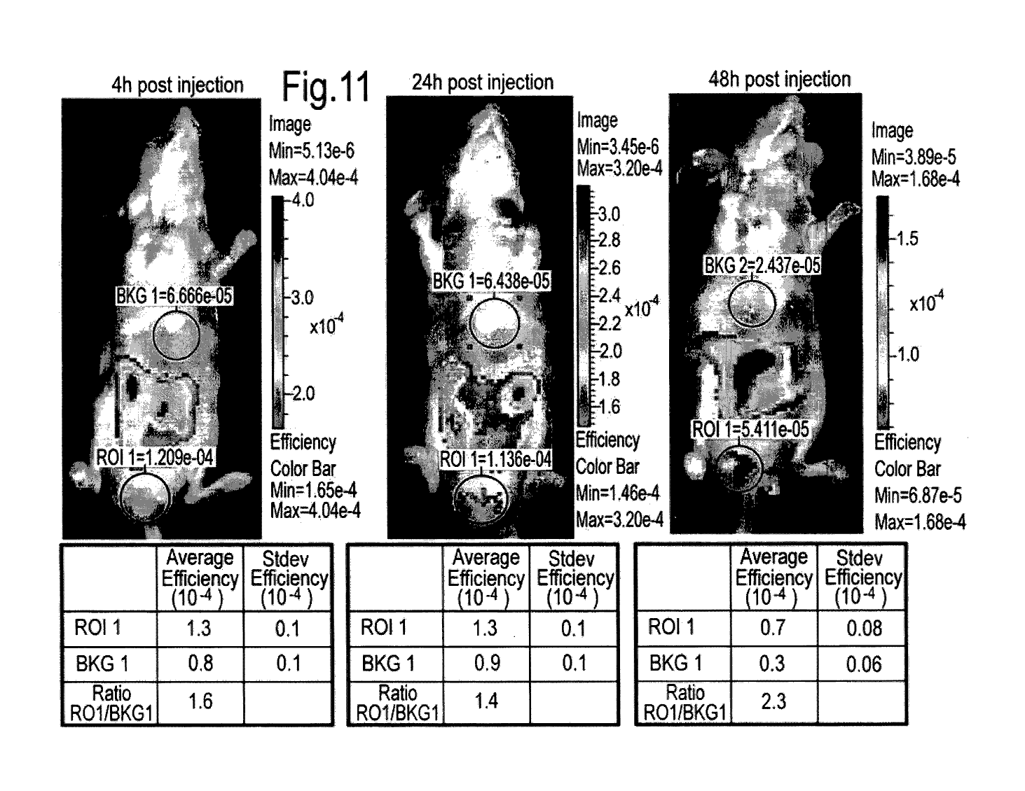

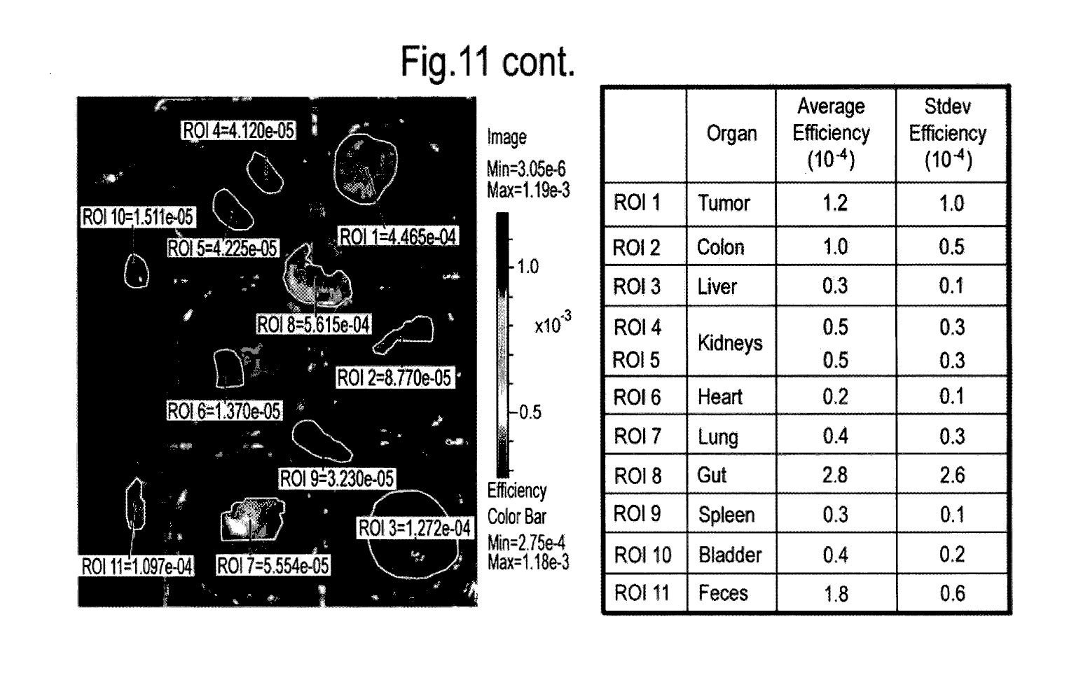

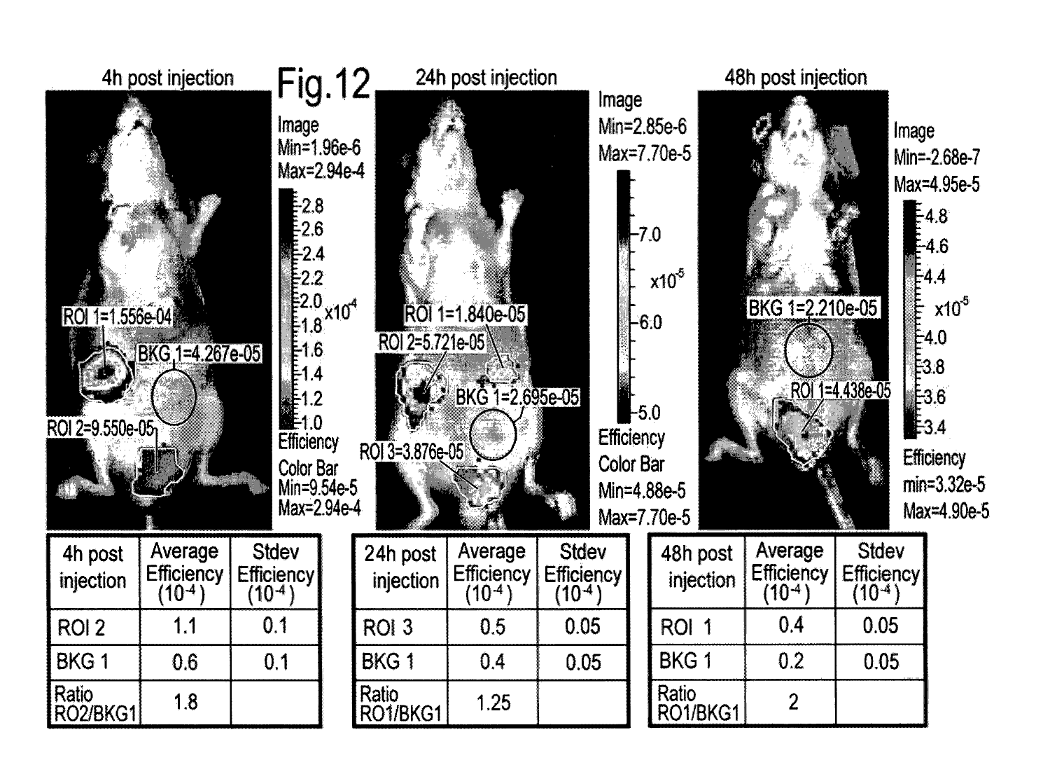

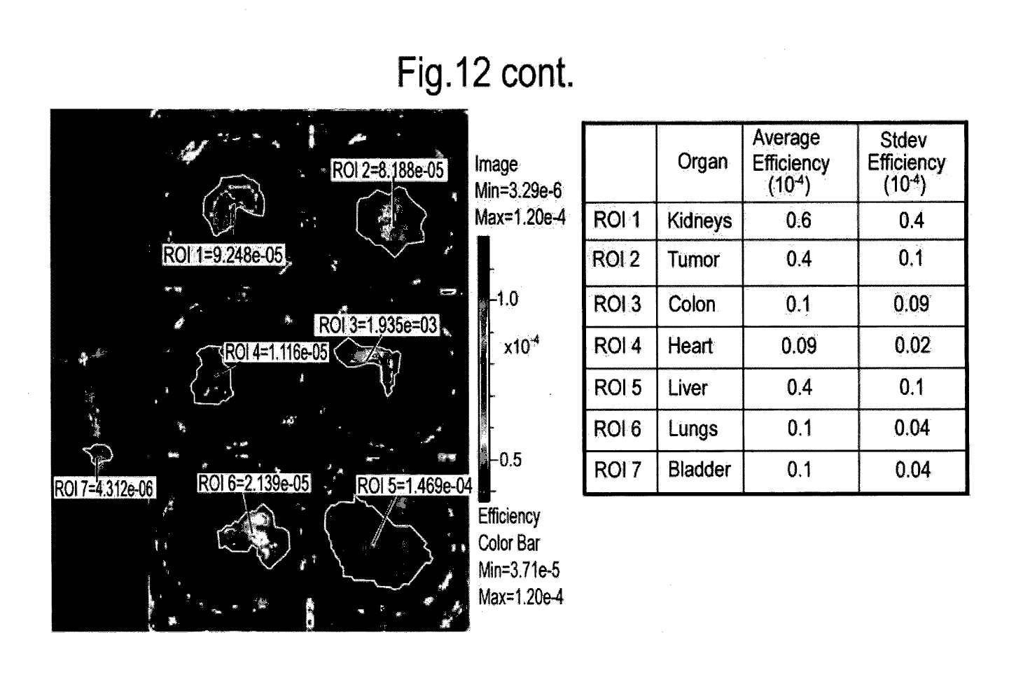

To test whether the presence of the EGFR targeting peptide could improve polymer accumulation and thus the detection of solid tumors in the GI tract, using the rectal tumor model described hereinabove, mice were injected with P-GE11-(GGFLGK-IR783) (FIG. 11) at a dose of 1 mg and the average fluorescence intensity measured was compared to that of the non-targeted degradable probe P-(GGFLGK-IR783)_{7.5}% at the same dose (FIG. 12). Whole body images were taken 4, 24 and 48 h post injection. Although differences in average fluorescence efficiency were observed at the tumor area, there was a significantly stronger fluorescent signal proximal to the abdominal area in mice injected with P-GE11-(GGFLGK-IR783). Mice were then sacrificed and ex vivo imaging of the organs was performed. In addition to the tumor labeling, the feces, stomach and the colon of mice were significantly fluorescent, most probably due to consumption of excreted feces containing IR-783-S-Ph-COOH that was eliminated during the experiments. This can also explain the fluorescent signal at the abdominal area that was found during whole body imaging. No significant difference was demonstrated after injection of targeted (P-GE11-(GGFLGK (SEQ ID NO: 11)-IR783)) and non-targeted probes (P-(GGFLGK (SEQ ID NO: 11)-IR783)_{7.5}%).

One of the problems associated with conventional low molecular imaging probes, is the limited T/B ratio.

In some embodiments, the polymers of this inventions show potential for actively target receptors overexpressed on tumors relative to normal tissues and undergoing optical activation within the malignant cells. As exemplified herein, and representing an embodiment of this invention, NIRF dye (IR-783-S-Ph-COOH) can serve as the optical reporter and if attached to the HPMA copolymer backbone via a tetrapeptide sequence (GFLG) it can be specificity cleaved by CB. The close spatial proximity of the multiple IR-783 molecules can result in quenching of fluorescence in the bound state. In addition two types of targeting peptides were subsequently attached to a synthetic copolymer for efficient tumoral targeting (C3-G12 and GE11 for binding Galectin-3 and EGFR, respectively). One embodied advantage of the synthesized polymeric probe over other low molecular reporters (e.g., isotopes, iodinated agents for radiograph) is that it can be “silenced” and “activated,” enabling the design of molecular with a “switch like behavior”.

In some embodiments, the potential for quenching results in a reduction of background “noise” by several orders of magnitude and a single enzyme can cleave multiple fluorophors resulting in efficient signal amplification. The use of the water soluble, biocompatible HPMA copolymer backbone provides additional embodied advantages. For example, the high molecular weight of the polymer can be manipulated to improve a passive accumulation in the tumor area due to EPR effect. Another embodied advantage of the use of HPMA copolymer based probes is that it can be easily conjugated to an imaging molecule or targeting moiety in a tailor-made fashion. Multiple targeting moieties on a single polymeric chain may increase in binding affinity between the receptors and the polymeric probe due to multivalent display of targeting ligands, that can act simultaneously at two or more receptors to markedly improve the binding affinity.

Targeting colorectal cells using two well known receptors galectin-3 and EGFR was demonstrated herein using embodied polymers of this invention.

The binding affinity of polymers bearing either carbohydrate (galactose) or short peptide (C3-G12) were compared as molecule for targeting galectin-3. Galactose and short peptide G3-C12 were conjugated to FITC labeled copolymer (designated as P-Gal-FITC and P-G3-C12-FITC respectively) and their binding affinity and intracellular fate in different CRC cells were analyzed by flow cytometry and confocal microscopy assays. Both targeting moieties were found to enhance the binding affinity of the copolymer to galactin-3 expressing cells. The bound copolymers were further internalized by galectin-3 and localized at lysosomal compartments. This lysosomotropism may initiate the release of imaging probes introducing degradable GFLG linkage essential for the optical activation of the NIR fluorescent molecule. Despite the lower percentage of G3-C12 peptide in the copolymer relative to galactose moiety (˜3 mol % and ˜10 mol % respectively), the binding of P-G3-C12-FITC copolymer to the galectin-3 positive cells was significantly higher compared to the P-Gal-FITC. Moreover, P-G3-C12-FITC was visualized more clearly by confocal microscopy when compared with P-Gal-FITC copolymer. These results indicate that G3-C12 peptide has superior ability to target FITC labeled copolymer to galectin-3 expressing CRC relative to galactose.

For targeting EGFR an embodied GE11-containing polymer was used.

Embodied polymeric probes were shown to detect solid tumors in vivo. Polymers with different molar percentages of IR-783-S-Ph-COOH dye (2.5%, 5% and 7.5%) were injected intravenously at various doses (2, 1, and 0.2 mg/mouse) and the animal's whole body was scanned at three different time points (4, 24 and 48 h post injection). The imaging probes were indeed found to detect solid tumors after IV administration. The results support the assumption that macromolecular imaging probes can passively accumulate in solid tumor due to EPR effect as soon as 4 hours post injection. This was true for all the different copolymers; P-GGFLGK-IR783 bearing 2.5 and 7.5 molar percentage of IR-783-S-Ph-COOH dye, with or without the GE11 targeting peptide.

An IR-783 labeled copolymer bearing GE11 as targeting moiety towards EGFR overexpressing cells when injected intravenously into mice bearing rectally implanted tumors derived from EGFR positive SW-480 cells, and subjected to whole body imaging revealed the accumulation of the polymeric probes in tumors.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A polymer characterized by the structure of formula 1:
wherein 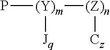
m, n, q and z indicate percentages of the respective monomer composition of the polymer, wherein m is between about 0.05%-50%, n is between 0.5 to 50%; and q and z are between about 0.5%-50%
C is a near infrared dye selected from the group consisting of Cy5, Cy5.5 Indocyanine green (ICG), IR783 and analogs thereof, covalently linked to the polymeric backbone.
J is a short peptide, monoscaccharide or oligosaccharide targeting moiety;
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm is a protease-cleavable linker, a pH-sensitive linker or an esterase-cleavable linker; and
P is a polymeric group comprising underivatized or derivatized monomers of N-(2-hydroxypropyl)methacrylamide (HPMA), N-methylacrylamide, N,N-dialkylacrylamides, acrylic acid, methacrylic acid, polyamino acids, polysaccharides; polymers containing polyethyleneoxide sequences and polyvinyl pyrrolidone-maleic anhydride polymers, polylactic-co-glycolic acid, dendrimers, polysaccharides, peptides, proteins, polymer-peptide conjugates or polymer-protein conjugates.

2. The polymer of claim 1, wherein said protease cleavable linker is cleavable by a lysosomal thiol-dependent protease.

3. The polymer of claim 2, wherein said protease cleavable linker is a tetra-peptide degradable spacer.

4. The polymer of claim 3, wherein said linker is Gly-Phe-Leu-Gly.

5. The polymer of claim 1, wherein said pH-dependent cleavable linker comprises a cis-aconityl, acetal or hydrazone moiety which undergoes pH-dependent hydrolysis following internailization within an acidic intracellular compartment.

6. The polymer of claim 1, wherein said carbohydrate targeting moiety is a monosaccharide, an oligosaccharide or a derivative thereof.

7. The polymer of claim 1, wherein said peptide targeting moiety is a monoclonal antibody or a fragment thereof, which binds to a specific cell surface marker.

8. The polymer of claim 7, wherein said cell surface marker is a cancer marker.

9. The polymer of claim 1, wherein Y is characterized by the structure of formulae IIa, or IIb or IIc as follows: 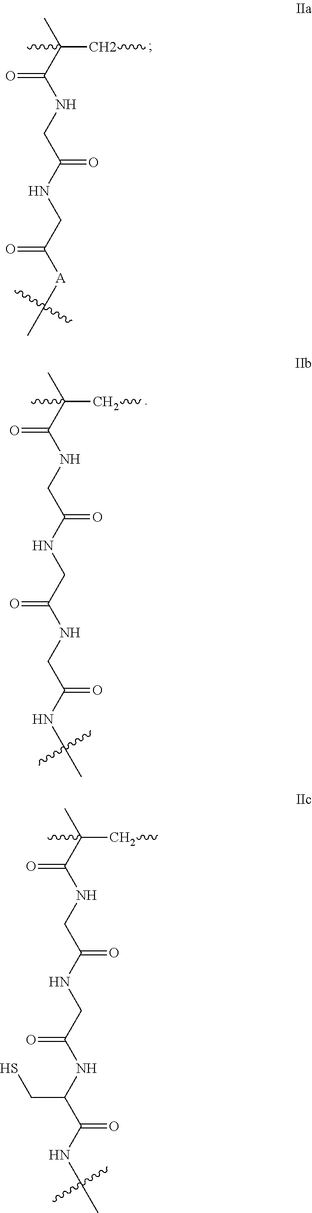
where A is an amine or an alcohol.

10. The polymer of claim 1, wherein the molecular weight of said polymer ranges between 15-60 kDa.

11. The polymer of claim 1, wherein said polymer is water soluble.

12. The polymer of claim 1, wherein said imaging agent is 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide.

13. The polymer of claim 1, wherein said polymer is represented by the structure of formula III:

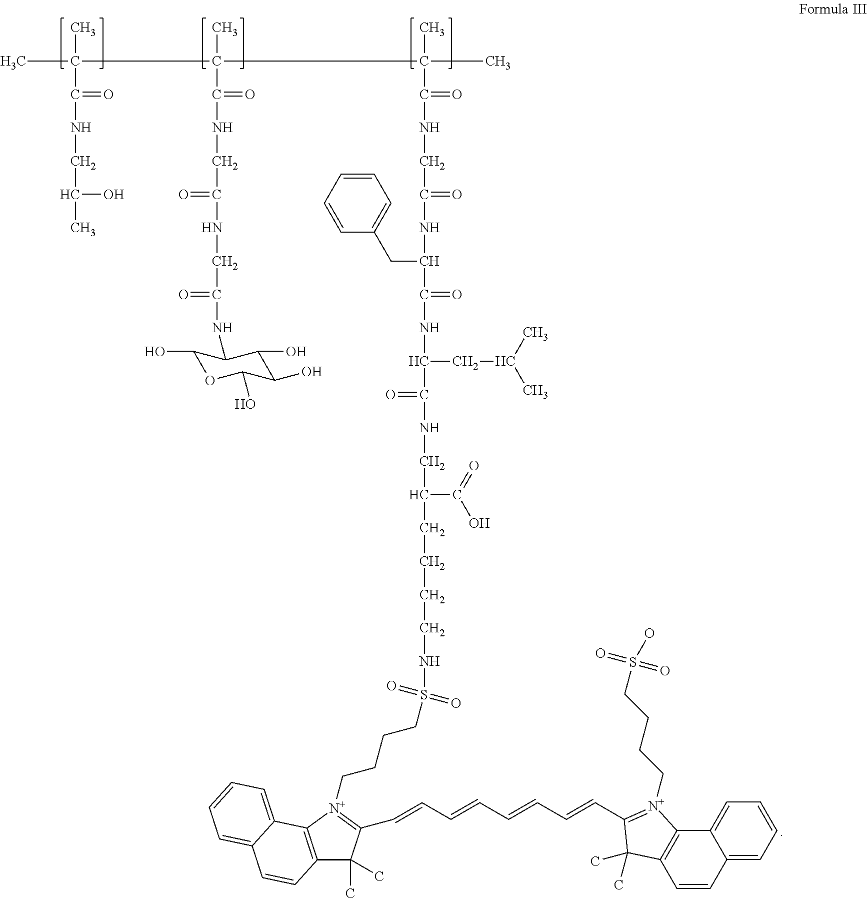

14. The polymer of claim 1, wherein said polymer is represented by the structure of formula IV:

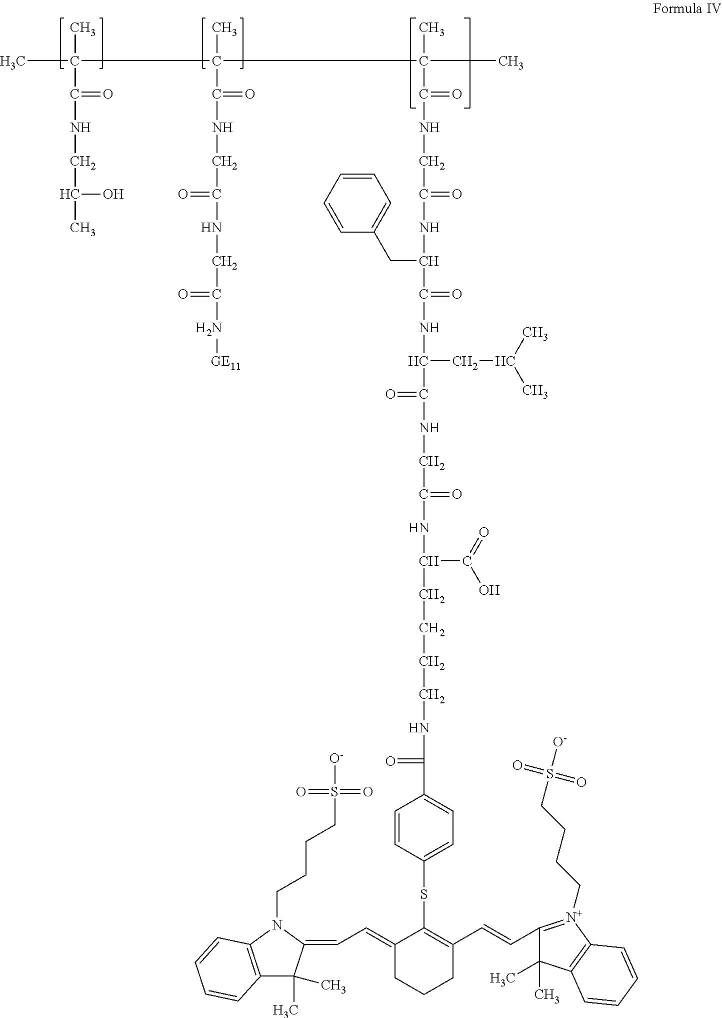

15. The polymer of claim 1, wherein J is derived from a galectin, EGF receptor or Muc-1 protein.

16. The polymer of claim 1, wherein J is an EPPT1 peptide.

17. A pharmaceutical composition comprising the polymer of claim 1.

18. The composition of claim 17, further comprising a carrier, diluent, lubricant, flow-aid, or a mixture thereof.

19. The composition of claim 17, wherein said composition is in the form of a pellet, a tablet, a capsule, a solution, a suspension, a dispersion, an emulsion, an elixir, a gel, an ointment, a cream, an aqueous solution or a suppository.

20. The composition of claim 17, wherein said composition is in the form of a capsule.

21. The composition of claim 17, wherein said composition is in a form suitable for oral, intravenous, intraarterial, intramuscular, intracranial, intranasal, subcutaneous, parenteral, transmucosal, transdermal, or topical administration.

22. The composition of claim 17, wherein said composition is a controlled release composition.

23. The composition of claim 17, wherein said composition is an immediate release composition.

24. The composition of claim 17, wherein said composition is a liquid dosage form.

25. The composition of claim 17, wherein said composition is a solid dosage form.

26. The composition of claim 17, further comprising an antineoplastic compound, an immunotherapeutic agent or a drug.

27. A method of imaging an inflammatory condition in a subject, said method comprising administering a polymer of claims 1 to said subject.

28. A method of imaging a disease associated with neovascularization in a subject, said method comprising administering a polymer of claim 1 to said subject.

29. A method of imaging a cancer or cancerous tissue in a subject, said method comprising the step of contacting said cancer or cancerous tissue with a polymer of claim 1.

30. The method of claim 29, wherein said polymer binds to receptors on neoplastic cells.

31. The method of claim 29, wherein, said neoplastic cell is derived from the lung, breast, prostate, colon or pancreas.

32. The method of claim 29, wherein said neoplastic cell is a carcinoma, sarcoma, lymphoma, or leukemia cell.

33. The method of claim 29, wherein said polymer is administered intra-tumorally.

34. The method of claims 29, further comprising the step of providing anti cancer therapy to imaged cancer or cancerous tissue in said subject.

35. The method of claim 34, wherein said anti-cancer therapy comprises surgery, chemotherapy, radiation or a combination thereof.

36. The method of claim 29, wherein said spacer undergoes cleavage induced by cysteine peptidases.

37. The method of claim 36, wherein said cysteine peptidase is cathepsin B.

38. The method of claim 36, wherein the source of said cathepsin B is the lysosomal compartments of tumor cells.

39. The method of claim 29, wherein said diagnosis comprises the detection of said tag moiety on said polymer.

40. The method of claim 29, wherein said detection of the tag moiety is an optical detection.


SEQUENCE LISTING (11 sequences)

SEQ ID NO: 1 - protein, 4 aa, Homo sapiens
GFLG

SEQ ID NO: 2 - protein, 6 aa, Homo sapiens
GGGGFG

SEQ ID NO: 3 - protein, 6 aa, Homo sapiens
GGGFLG

SEQ ID NO: 4 - protein, 4 aa, Homo sapiens
GGEE

SEQ ID NO: 5 - protein, 6 aa, Homo sapiens
GGGLFG

SEQ ID NO: 6 - protein, 4 aa, Homo sapiens
GGKK

SEQ ID NO: 7 - protein, 12 aa, Homo sapiens
YHWYGYTPQNVI

SEQ ID NO: 8 - protein, 16 aa, Homo sapiens
ANTPCGPYTHDCPVKR

SEQ ID NO: 9 - protein, 12 aa, Homo sapiens
YHWYGYTPQNVI

SEQ ID NO: 10 - protein, 15 aa, Homo sapiens
YCAREPPTRTFAYWG

SEQ ID NO: 11 - protein, 6 aa, Homo sapiens
GGFLGK